(19) United States
(12) Patent Application Publication
(10) Pub. No.: US 2025/0084471 A1
(43) Pub. Date: Mar. 13, 2025

(54) ALPHA-HEMOLYSIN VARIANTS FORMING NARROW CHANNEL PORES AND USES THEREOF

(71) Applicant: Roche Sequencing Solutions, Inc., Pleasanton, CA (US)

(72) Inventors: Aruna Shankaranarayanan Ayer, Santa Clara, CA (US); Adolfo Vargas, San Mateo, CA (US); Rongxin Nie, San Jose, CA (US); Seyedeh Narges Molavi Arabshahi, Santa Clara, CA (US)

(21) Appl. No.: 18/580,302

(22) PCT Filed: Jul. 19, 2022
(86) PCT No.: PCT/EP2022/070110

§ 371 Date: Jan. 18, 2024

Related U.S. Application Data
(60) Provisional application No. 63/224,282, filed on Jul. 21, 2021.

(51) Int. Cl. C12Q 1/6869; C07K 14/31; C12N 15/10

(52) U.S. Cl. CPC C12Q 1/6869; C07K 14/31; C12N 15/1096

(57) ABSTRACT

Described herein are alpha-hemolysin nanopores having relatively narrow channels and D127G and D128K substitutions relative to SEQ ID NO: 1. The narrow channel reduces the extent to which the nucleic acid template threads through the nanopore, while the D127G and D128K substitutions improve the lifetime and arrival rate of the narrow channel pores. Also disclosed herein are polypeptides for forming such nanopores, systems comprising such nanopores, and methods of making and using such nanopores.

### TECHNICAL FIELD

Disclosed are compositions and methods relating to variants of Staphylococcal aureaus alpha-hemolysin polypeptides. The alpha-hemolysin (alpha hemolysin) variants are useful, for example, as a nanopore component in a device for determining polymer sequence information.

### BACKGROUND

Hemolysins are members of a family of protein toxins that are produced by a wide variety of organisms. Some hemolysins, for example alpha hemolysins, can disrupt the integrity of a cell membrane (e.g., a host cell membrane) by forming a pore or channel in the membrane. Pores or channels that are formed in a membrane by pore forming proteins can be used to transport certain polymers (e.g., polypeptides or polynucleotides) from one side of a membrane to the other.

Alpha-hemolysin (also referred to as α-hemolysin, α-HL, α-HL or alpha-HL) is a self-assembling toxin which forms a channel in the membrane of a host cell. alpha hemolysin has become a principal component for the nanopore sequencing community. It has many advantageous properties including high stability, self-assembly, and a pore diameter which is wide enough to accommodate single stranded DNA but not double stranded DNA (Kasianowicz et al., 1996).

Previous work on DNA detection in the α-HL pore has focused on analyzing the ionic current signature as DNA translocates through the pore (Kasianowicz et al., 1996, Akeson et al., 1999, Meller et al., 2001), a very difficult task given the translocation rate (˜1 nt/μs at 100 mV) and the inherent noise in the ionic current signal. Higher specificity has been achieved in nanopore-based sensors by incorporation of probe molecules permanently tethered to the interior of the pore (Howorka et al., 2001a and Howorka et al., 2001b; Movileanu et al., 2000).

Wild-type alpha hemolysin results in significant number of deletion errors, i.e. bases are not measured. Therefore, numerous efforts have been made at improving alpha hemolysin nanopores for use in tag-based sequencing-by-synthesis (SBS), Examples include US 2017-0088588 A1, US 2017-0088890 A1, US 2017-0306397 A1, US 2018-0002750 Al, and US 2018-0002750 A1. A need remains, however, for alpha hemolysin nanopores with improved properties.

### BRIEF SUMMARY OF THE INVENTION

Variants of -staphylococcal alpha hemolysin polypeptides containing an amino acid variation useful for generating nanopores that can be used in tag-based sequencing-by-synthesis reactions are disclosed. The variant polypeptides disclosed herein may be used to prepare heptameric nanopores that have relatively narrow constriction sites and longer pore lifetime when compared to pores formed from reference alpha hemolysin polypeptides.

In an aspect, an alpha-hemolysin (alpha hemolysin) polypeptide comprising at least one narrow channel α-hemolysin (alpha hemolysin) subunit is provided, said subunit comprising D127G and D128K substitutions relative to SEQ ID NO: 1. In some embodiments, the amino acid residue corresponding to E111 and/or K147 of SEQ ID NO: 1 is selected from the group consisting of glutamic acid, lysine, arginine, and glutamine. In some embodiments, the amino acid residue corresponding to E111 and/or K147 of SEQ ID NO: 1 is selected from the group consisting of glutamic acid and lysine. In some embodiments, the narrow channel alpha hemolysin subunit comprises either or both of E111 and K147 (i.e. wild-type residues at those positions relative to SEQ ID NO: 1). In some embodiments, the amino acid residue corresponding to M113 of SEQ ID NO: 1 is selected from the group consisting of leucine, isoleucine, valine, and methionine. In some embodiments, the amino acid residue corresponding to M113 of SEQ ID NO: 1 is methionine (i.e. wild-type residue at that position relative to SEQ ID NO: 1). In some embodiments, the narrow channel alpha hemolysin subunit comprises each of E111, M113, and K147 (i.e. wild-type residues at those positions relative to SEQ ID NO: 1). For example, the narrow channel alpha hemolysin subunit may comprise an amino acid sequence having at least 75%, 80%, 90%, 95%, 98%, or more identity to

SEQ ID NO: 1, wherein the amino acid sequence comprises a glutamic acid residue at a position corresponding to E111 of SEQ ID NO: 1, a methionine residue at a position corresponding to M113 of SEQ ID NO: 1, a lysine residue at a position corresponding to K147 of SEQ ID NO:1, a D127G substitution relative to SEQ ID NO: 1, and a D128K substitution relative to SEQ ID NO: 1. As another example, the narrow channel alpha hemolysin subunit may comprise an amino acid sequence having at least 75%, 80%, 90%, 95%, 98%, or more identity to SEQ ID NO: 2, wherein the amino acid sequence comprises each of G127, K128, E111, M113, and K147 of SEQ ID NO: 2. As another example, the narrow channel alpha hemolysin subunit comprises an amino acid sequence having at least 75%, 80%, 90%, 95%, 98%, or more identity to SEQ ID NO: 3, wherein the amino acid sequence comprises each of G127, K128, E111, M113, and K147 of SEQ ID NO: 3. As another example, the narrow channel alpha hemolysin subunit comprises or consists of an amino acid sequence selected from the group consisting of SEQ ID NO: 2 and SEQ ID NO: 3. As another example, the narrow channel alpha hemolysin subunit comprises an amino acid sequence having at least 75%, 80%, 90%, 95%, 98%, or more identity to SEQ ID NO: 4, wherein the amino acid sequence comprises N111E, A113M, and N147K substitutions relative to SEQ ID NO: 4 and further comprises G127 and K128 of SEQ ID NO: 4. As another example, the narrow channel alpha hemolysin subunit comprises an amino acid sequence having at least 75%, 80%, 90%, 95%, 98%, or more identity to SEQ ID NO: 5, wherein the amino acid sequence comprises N111E, A113M, N147K, and G128K substitutions relative to SEQ ID NO: 5 and further comprises G127 of SEQ ID NO: 5. As another example, the narrow channel alpha hemolysin subunit comprises an amino acid sequence having at least 75%, 80%, 90%, 95%, 98%, or more identity to SEQ ID NO: 6, wherein the amino acid sequence comprises a D127G substitution relative to SEQ ID NO: 6, a D128K substitution relative to SEQ ID NO: 6, a glutamic acid residue at a position corresponding to E111 of SEQ ID NO: 6, a methionine residue at a position corresponding to M113 of SEQ ID NO: 6, and a lysine residue at a position corresponding to K147 of SEQ ID NO: 6. As another example, the narrow channel alpha hemolysin subunit comprises an amino acid sequence having at least 75%, 80%, 90%, 95%, 98%, or more identity to SEQ ID NO: 7, wherein the amino acid sequence comprises a D127G substitution relative to SEQ ID NO: 7, a D128K substitution relative to SEQ ID NO: 7, a glutamic acid residue at a position corresponding to E111 of SEQ ID NO: 7, a methionine residue at a position corresponding to M113 of SEQ ID NO: 7, and a lysine residue at a position corresponding to K147 of SEQ ID NO: 7. As another example, the narrow channel alpha hemolysin subunit comprises an amino acid sequence having at least 75%, 80%, 90%, 95%, 98%, or more identity to SEQ ID NO: 8, wherein the amino acid sequence comprises a D127G substitution relative to SEQ ID NO: 8, a D128K substitution relative to SEQ ID NO: 8, a glutamic acid residue at a position corresponding to E111 of SEQ ID NO: 8, a methionine residue at a position corresponding to M113 of SEQ ID NO: 8, and a lysine residue at a position corresponding to K147 of SEQ ID NO: 8.

Narrow channel alpha hemolysin nanopores are also provided, said nanopores comprising at least 6 narrow channel alpha hemolysin subunits comprising D127G and D128K substitutions relative to SEQ ID NO: 1. The nanopores have the following properties: (a) a constriction site that is narrower than nanopore P-0304; and (b) increased lifetime relative to nanopore P-0031. In certain embodiments, the narrow channel alpha hemolysin nanopore described herein is bound to a DNA polymerase, such as via a covalent bond. In certain exemplary embodiments, the narrow channel alpha hemolysin nanopore is a 6:1 nanopore, and the DNA polymerase is attached to the “1” component.

In certain example aspects, also provided are nucleic acids encoding any of the narrow channel alpha hemolysin variant polypeptides described herein. For example, the nucleic acid sequence can be derived from Staphylococcus aureus αHL (SEQ ID NO: 9). Also provided, in certain example aspects, are vectors that include an any such nucleic acids encoding any one of the hemolysin variants described herein. Also provided is a host cell that is transformed with the vector.

In certain example aspects, provided is a method of detecting and/or identifying a target nucleic acid molecule using the disclosed narrow channel alpha-hemolysin nanopores. The method includes, for example, providing a chip comprising a nanopore assembly as described herein in a membrane that is disposed adjacent or in proximity to a sensing electrode. The method then includes detecting tagged nucleotides using the nanopore during the synthesis of a complementary strand of the target nucleic acid molecule.

Other objects, features, and advantages of the present invention will become apparent from the following detailed description. It should be understood, however, that the detailed description and specific examples, while indicating embodiments of the invention, are given by way of illustration only, since various changes and modifications within the scope and spirit of the invention will become apparent to one skilled in the art from this detailed description.

### BRIEF DESCRIPTION OF THE DRAWINGS

FIG. 1 depicts two sequencing runs with potential threading issues. (A) illustrates a sequencing run with clear open channel levels 101, tag levels 102a-102d, and a persistent background level 103 likely caused by template threading. (B) illustrates a sequencing run with significant background noise 103 and sequencing abrogation 104 likely caused by template threading.

FIG. 2 is a graph of arrival rate (X-axis) versus pore lifetime (Y-axis) of 4 different pores: P-0031, P-0304, P-0411, and P-0414.

FIG. 3 is a bar graph showing fraction of threaded pores using a wide channel (P-0304) versus a narrow channel (P-0411 and P-0414) alpha hemolysin nanopore.

FIG. 4 is a sequence alignment between the subunits disclosed at Table 5.

### DETAILED DESCRIPTION

The invention will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this invention. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

Numeric ranges are inclusive of the numbers defining the range. The term about is used herein to mean plus or minus ten percent (10%) of a value. For example, “about 100” refers to any number between 90 and 110.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention, which can be had by reference to the specification as a whole. Accordingly, the terms defined immediately below are more fully defined by reference to the specification as a whole.

### I. Definitions

Alpha-hemolysin: As used herein, “alpha-hemolysin,” “α-hemolysin,” “a-HL” and “alpha hemolysin” are used interchangeably and refer to polypeptides expressed from the hly gene of Staphylococcus aureus.

Alpha-hemolysin nanopore: As used herein, an “alpha-hemolysin nanopore” refers to a nanopore formed from 7 alpha-hemolysin subunits.

Alpha-hemolysin polypeptide: As used herein, an “alpha-hemolysin polypeptide” refers to any polypeptide that comprises at least one alpha-hemolysin subunit.

Alpha-hemolysin subunit: As used herein, an “alpha-hemolysin subunit” refers to SEQ ID NO: 1 and variants thereof that are capable of self-assembling into a heptameric nanopore.

Amino acid: As used herein, the term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” or “non-natural amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical without adversely affecting their activity. Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide. It should be noted that all amino acid residue sequences are represented herein by formulae whose left and right orientation is in the conventional direction of amino-terminus to carboxy-terminus.

Arrival Rate: As used herein, the “arrival rate” of an alpha hemolysin nanopore is a measure of frequency with which the alpha hemolysin nanopore captures the tag of a biotinylated tag molecule. For example, arrival rate can be determined by obtaining a chip having a plurality of the pore of interest inserted in the bilayer, flowing a streptavidin-biotin-TAG across the chip, and measuring the average time between capture events at each of the plurality of pores (typically at a very low AC modulation frequency, such as ˜50 Hz). The arrival rate is the average time between events across all pores.

Base Pair (bp): As used herein, base pair refers to a partnership of adenine (A) with thymine (T), adenine (A) with uracil (U) or of cytosine (C) with guanine (G) in a double stranded nucleic acid.

Complementary: As used herein, the term “complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide.

Concatenated alpha hemolysin polypeptide: An alpha-hemolysin polypeptide that includes multiple alpha-hemolysin subunits separated from one another by one or more flexible linker sequences. Exemplary methods of generating concatenated alpha hemolysin polypeptides and considerations for doing so are disclosed by, for example, Hammerstein and US 2017-0088890 A1.

Expression cassette: An “expression cassette” or “expression vector” is a nucleic acid construct generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

Heterologous: A “heterologous” nucleic acid construct or sequence has a portion of the sequence which is not native to the cell in which it is expressed. Heterologous, with respect to a control sequence, refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, transformation, microinjection, electroporation, or the like. A “heterologous” nucleic acid construct may contain a control sequence/DNA coding sequence combination that is the same as, or different from a control sequence/DNA coding sequence combination found in the native cell.

Host cell: By the term “host cell” is meant a cell that contains a vector and supports the replication, and/or transcription or transcription and translation (expression) of the expression construct. Host cells for use in the present invention can be prokaryotic cells, such as E. coli or Bacillus subtilus, or eukaryotic cells such as yeast, plant, insect, amphibian, or mammalian cells. In general, host cells are prokaryotic, e.g., E. coli.

Isolated: An “isolated” molecule is a nucleic acid molecule that is separated from at least one other molecule with which it is ordinarily associated, for example, in its natural environment. An isolated nucleic acid molecule includes a nucleic acid molecule contained in cells that ordinarily express the nucleic acid molecule, but the nucleic acid molecule is present extrachromasomally or at a chromosomal location that is different from its natural chromosomal location.

Lifetime: As used herein, the “lifetime” of a species of alpha hemolysin nanopore is a measure of the percentage of alpha hemolysin nanopores that remain capable of capturing the tag of a biotinylated tag molecule for a 1 hour period on a nanopore sequencing array. For example, lifetime can be determined by obtaining a chip having a plurality of the pore of interest inserted in the bilayer, flowing the streptavidin-biotin-TAG across the chip, and tracking the activity of all of the individual nanopores on the chip over a 1 hour period. The lifetime of the pore species is the percentage of pores that remain active for the entire 1 hour period.

Mutation: As used herein, the term “mutation” refers to a change introduced into a parental sequence, including, but not limited to, substitutions, insertions, and/or deletions (including truncations). The consequences of a mutation include, but are not limited to, the creation of a new character, property, function, phenotype or trait not found in the protein encoded by the parental sequence.

Nanopore: The term “nanopore,” as used herein, generally refers to a pore, channel or passage formed or otherwise provided in a membrane. A membrane may be an organic membrane, such as a lipid bilayer, or a synthetic membrane, such as a membrane formed of a polymeric material. The membrane may be a polymeric material. The nanopore may be disposed adjacent or in proximity to a sensing circuit or an electrode coupled to a sensing circuit, such as, for example, a complementary metal-oxide semiconductor (CMOS) or field effect transistor (FET) circuit. In some examples, a nanopore has a characteristic width or diameter on the order of 0.1 nanometers (nm) to about 1000 nm. Some nanopores are proteins. Alpha-hemolysin is an example of a nanopore-forming polypeptide.

Narrow channel alpha-hemolysin nanopore: As used herein, a narrow channel alpha hemolysin nanopore is an alpha hemolysin nanopore that comprises at least 6 narrow channel alpha hemolysin subunits.

Narrow channel alpha-hemolysin polypeptide: As used herein, a narrow channel alpha hemolysin polypeptide is an alpha hemolysin polypeptide that comprises at least 1 narrow channel alpha hemolysin subunit.

Narrow channel alpha-hemolysin subunit: As used herein, a narrow channel alpha hemolysin subunit is an alpha hemolysin subunit that, when aligned with SEQ ID NO: 1, has: (a) an amino acid at a position corresponding to E111 of SEQ ID NO: 1 that has a sidechain that is longer than the side chain of asparagine (such as glutamic acid, lysine, arginine, or glutamine), (b) an amino acid at a position corresponding to K147 of SEQ ID NO: 1 that has a sidechain that is longer than the side chain of asparagine (such as glutamic acid, lysine, arginine, or glutamine), and/or (c) an amino acid at a position corresponding to M113 of SEQ ID NO: 1 that has a sidechain that is longer than the side chain of alanine (such as leucine, isoleucine, valine, and methionine).

Nucleic Acid Molecule: The term “nucleic acid molecule” includes RNA, DNA and cDNA molecules. It will be understood that, as a result of the degeneracy of the genetic code, a multitude of nucleotide sequences encoding a given protein such as alpha-hemolysin and/or variants thereof may be produced. The present invention contemplates every possible variant nucleotide sequence, encoding variant alpha-hemolysin, all of which are possible given the degeneracy of the genetic code.

Percent identity: The term “% identity” refers to the level of nucleic acid or amino acid identity between the nucleic acid sequence that encodes any one of the inventive polypeptides or the inventive polypeptide's amino acid sequence, when aligned using a sequence alignment program. For example, as used herein, 80% identity embraces homologues of a given sequence having greater than 80% identity over a length of the given sequence. Exemplary levels of identity include, but are not limited to, 75%, 80%, 85%, 90%, 95%, 98% or more identity to a given sequence, e.g., the coding sequence for any one of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is may be used for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, e.g., Altschul, S. F., et al., Nucleic Acids Res. 25:3389-3402, 1997.) An alignment of selected sequences in order to determine “% identity” between two or more sequences, may be performed using for example, the CLUSTAL-W program in MacVector version 13.0.7, operated with default parameters, including an open gap penalty of 10.0, an extended gap penalty of 0.1, and a BLOSUM 30 similarity matrix.

Promoter: As used herein, the term “promoter” refers to a nucleic acid sequence that functions to direct transcription of a downstream gene. The promoter will generally be appropriate to the host cell in which the target gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

Purified: As used herein, “purified” means that a molecule is present in a sample at a concentration of at least 95% by weight, or at least 98% by weight of the sample in which it is contained.

Tag: As used herein, the term “tag” refers to a nanopore-detectable moiety that may be atoms or molecules, or a collection of atoms or molecules. A tag may provide an optical, electrochemical, magnetic, or electrostatic (e.g., inductive, capacitive) signature, which signature may be detected with the aid of a nanopore. Typically, when a nucleotide is attached to the tag it is called a “Tagged Nucleotide.”

Variant: As used herein, the term “variant” refers to a polypeptide which displays altered primary amino acid sequence when compared to a wild-type polypeptide from which it is derived.

Variant alpha hemolysin polypeptide: The term “variant alpha-hemolysin polypeptide” or “variant αHL polypeptide” means an alpha-hemolysin polypeptide comprising at least one variant alpha hemolysin subunit.

Variant alpha hemolysin subunit: The term “variant alpha-hemolysin” or “variant αHL” means an alpha-hemolysin polypeptide with one or more substitutions, insertions, or deletions relative to SEQ ID NO: 1

Variant narrow channel alpha hemolysin nanopore: The term “variant narrow channel alpha hemolysin nanopore” means an narrow channel alpha-hemolysin nanopore in which at least 1 of the 6 narrow channel alpha hemolysin subunits is a variant narrow channel alpha hemolysin subunits.

Variant narrow channel alpha hemolysin polypeptide: The term “variant narrow channel alpha hemolysin polypeptide” is an alpha hemolysin polypeptide that comprises at least 1 variant narrow channel alpha hemolysin subunit.

Variant narrow channel alpha hemolysin subunit: The term “variant narrow channel alpha hemolysin subunit” means an narrow channel alpha-hemolysin subunit with one or more substitutions, insertions, or deletions relative to SEQ ID NO: 1.

Vector: As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art.

Wild-type alpha hemolysin: As used herein, the term “wild-type alpha hemolysin” refers to an alpha hemolysin subunit comprising SEQ ID NO: 1.

### II. Nomenclature

In the present description and claims, the conventional one-letter and three-letter codes for amino acid residues are used.

For ease of reference, variants of the application are described by use of the following nomenclature: Original amino acid(s); position(s); substituted amino acid(s). According to this nomenclature, for instance, the substitution of a valine by a lysine in position 149 is shown as:

Val149Lys or V149K
Multiple mutations are separated by plus signs, such as:

Ala1Lys+Asn47Lys+Glu287Arg or A1K+N47K+E287R

representing mutations in positions 1, 47, and 287 substituting lysine for alanine, lysine for asparagine, and arginine for glutamic acid, respectively. Spans of amino acid substitutions are represented by a dash, such as a span of glycine residues from residue 127 to 131 being: 127-131Gly or 127-133G.

### III. Development Background

A “wide channel” alpha-hemolysin nanopore is a nanopore in which one or more of the amino acids forming the constriction site have been modified to residues having short side chains relative to wild-type alpha-hemolysin. This provides a wider diameter at the constriction site than pores having the native residues, which allows tags to flow more freely through the beta barrel. Table 1 lists the solvent-facing amino acid residues of SEQ ID NO: 1 that form the channel. “#” indicates the position within SEQ ID NO: 1, “AA” indicates the amino acid at the recited position of SEQ ID NO: 1, and “Location” indicates the sub-region of the alpha hemolysin nanopore at which the amino acid is located.

TABLE 1

| # | AA | Location |
|---|---|---|
| 1 | Ala | Entrance |
| 2 | Asp | Entrance |
| 3 | Ser | Entrance |
| 4 | Asp | Entrance |
| 5 | Ile | Entrance |
| 6 | Asn | Entrance |
| 7 | Ile | Entrance |
| 8 | Lys | Entrance |
| 9 | Thr | Entrance |
| 10 | Gly | Entrance |
| 11 | Thr | Entrance |
| 12 | Thr | Entrance |
| 13 | Asp | Entrance |
| 14 | Ile | Entrance |
| 15 | Gly | Entrance |
| 16 | Ser | Entrance |
| 17 | Asn | Entrance |
| 18 | Thr | Entrance |
| 19 | Thr | Entrance |
| 20 | Val | Entrance |
| 21 | Lys | Entrance |
| 22 | Thr | Entrance |
| 46 | Lys | Entrance |
| 47 | Asn | Entrance |
| 106 | Ser | Entrance |
| 111 | Glu | Constriction |
| 113 | Met | Constriction |
| 115 | Thr | Beta Barrel |
| 117 | Thr | Beta Barrel |
| 119 | Gly | Beta Barrel |
| 121 | Asn | Beta Barrel |
| 123 | Asn | Beta Barrel |
| 125 | Thr | Beta Barrel |
| 129 | Thr | Exit |
| 130 | Gly | Exit |
| 131 | Lys | Exit |
| 133 | Gly | Beta Barrel |
| 135 | Leu | Beta Barrel |
| 137 | Gly | Beta Barrel |
| 139 | Asn | Beta Barrel |
| 141 | Ser | Beta Barrel |
| 143 | Gly | Beta Barrel |
| 145 | Thr | Beta Barrel |
| 147 | Lys | Constriction |
| 149 | Val | Entrance |
| 239 | Ser | Entrance |

BLANK

As can be seen, three amino acids make up the constriction site: E111, M113, and K147. In the classic “wide channel” alpha-hemolysin, both E111 and K147 are modified to asparagine (i.e. E111N and K147N substitutions relative to SEQ ID NO: 1) while M113 is modified to alanine (M113A substitution relative to SEQ ID NO: 1).

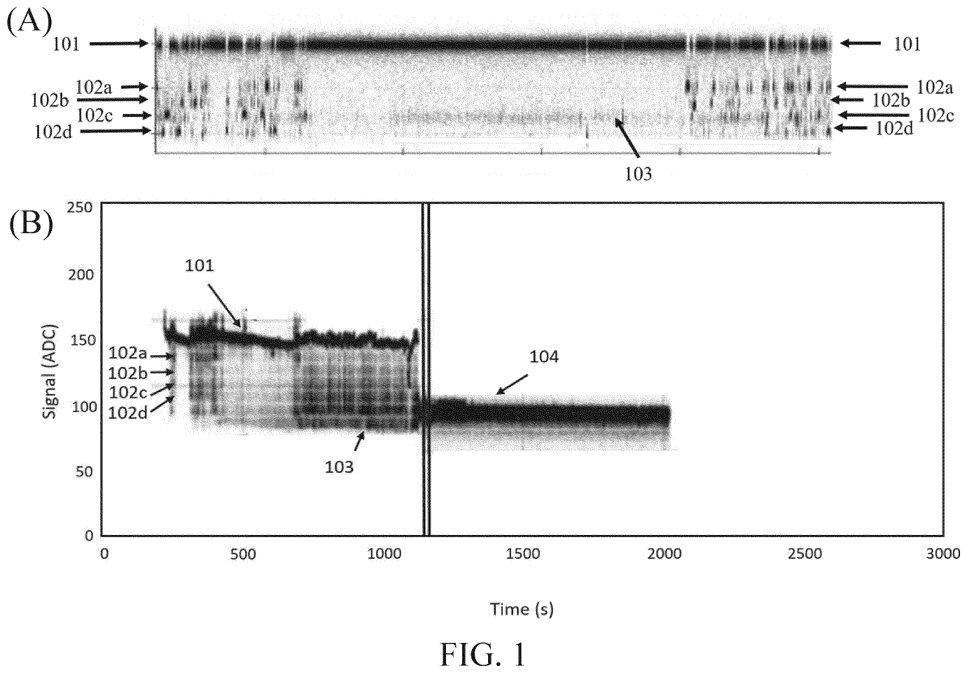

While wide channel alpha hemolysin pores typically have relatively high arrival rates, they do have some limitations. FIG. 1 illustrates two tag-based sequencing-by-synthesis (SBS) run using a wide channel α-hemolysin nanopore.

The dark band at the top is the open channel level 101 and a tag occupying the channel of the nanopore is recorded as a change in signal (in this case, conductance level) relative to open channel, with different tags resulting in different changes in signal 102a-102d. However, a persistent background band is frequently observed 103, which can result in convolution of tag signals that increases as the threading rate increases. Additionally, abrogation of sequencing activity can also be observed 104, as illustrated at (B). Both issues limit the throughput and accuracy of tag-based SBS. Without being bound by theory, the aberrant pattern may result at least in part from threading of the template nucleic acid and/or primer into the nanopore. It is believed that the background level is caused by the template and/or primer partially inserting into and ejecting from the nanopore, while the abrogation is caused by the template or primer threading completely through the nanopore.

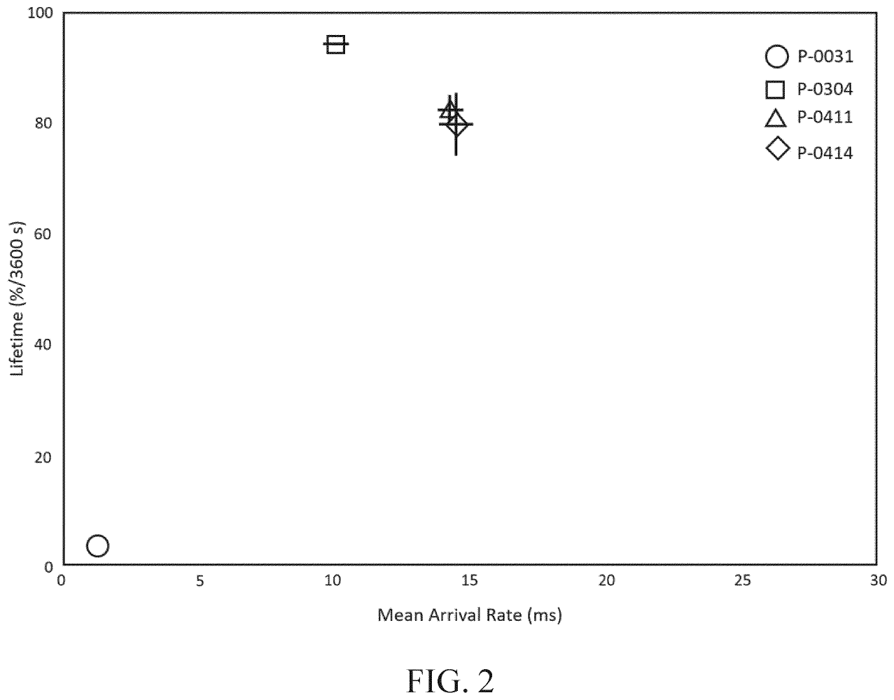

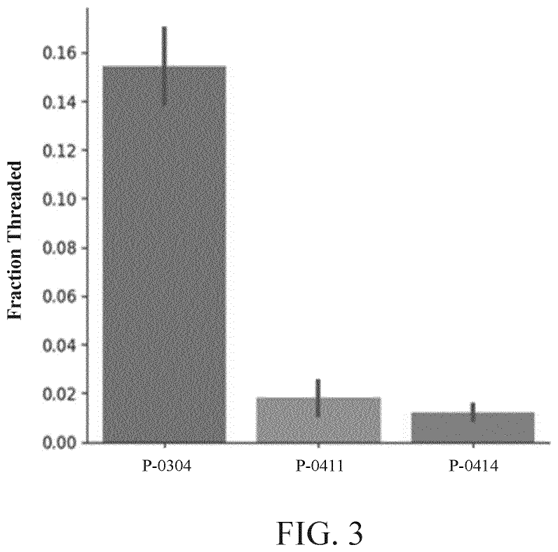

The present disclosure demonstrates that pairing a narrow channel alpha hemolysin nanopore with D127G and D128K substitutions results in relatively long lifetimes and acceptable arrival rates (FIG. 2) while at the same time significantly reducing the number of pores exhibiting the threading phenomenon (FIG. 3).

### IV. Polypeptides Comprising One or More Variant Narrow Channel Alpha-Hemolysin Subunit(s)

In one aspect, an isolated polypeptide is provided comprising, consisting essentially of, or consisting of a variant narrow channel alpha-hemolysin subunit, said subunit comprising D127G and D128K substitutions relative to SEQ ID NO: 1. The variant narrow channel alpha hemolysin subunits generally have at least the following characteristics:
(a) at least 75% identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8;
(b) a D127G substitution relative to SEQ ID NO: 1;
(c) a D128K substitution relative to SEQ ID NO: 1; and
(d) one or more of the following:
   (d1) an amino acid at a position corresponding to E111 of SEQ ID NO: 1 that has a sidechain that is longer than the side chain of asparagine (such as glutamic acid, lysine, arginine, or glutamine),
(d2) an amino acid at a position corresponding to K147 of SEQ ID NO: 1 that has a sidechain that is longer than the side chain of asparagine (such as glutamic acid, lysine, arginine, or glutamine), and/or
(d3) an amino acid at a position corresponding to M113 of SEQ ID NO: 1 that has a sidechain that is longer than the side chain of alanine (such as leucine, isoleucine, valine, and methionine).
   The combination of the substitutions at D127 and D128 relative to SEQ ID NO: 1 with longer amino acids at the constriction site reduce template threading relative to similar pores having a wide channels (such as pores that comprise E111N, M113A, and K147N), while simultaneously improving the lifetime of the resulting pores and having acceptable arrival rates.

In some of the embodiments described herein, the variant narrow channel alpha hemolysin nanopores are characterized according to their “threaded rate.” In this context, the “threaded rate” shall mean the percentage of 6:1 narrow channel alpha hemolysin nanopores with high quality reads (HQRs) that exhibit a threaded state, wherein the “6” component is the variant narrow channel alpha hemolysin subunit and the “1” component” is subunit G2043. The percentage of pores with the threaded state can be calculated as described in Example 5. In some embodiments, the variant narrow channel alpha hemolysin nanopores have a threaded rate of less than 15%. In some embodiments, the variant narrow channel alpha hemolysin nanopores have a threaded rate of less than 10%. In some embodiments, the variant narrow channel alpha hemolysin nanopores have a threaded rate of less than 5%. In some embodiments, the variant narrow channel alpha hemolysin nanopores have a threaded rate of less than 2%.

In some of the embodiments described herein, the variant narrow channel alpha hemolysin nanopores are characterized according to their “% lifetime.” In this context, the “% lifetime” shall mean the percentage of 6:1 narrow channel alpha hemolysin nanopores that remain active to a T40-tagged Streptavidin after 1 hour exposure to a 350 mV sequencing waveform, wherein the “6” component is the variant narrow channel alpha hemolysin subunit and the “1” component” is subunit G2043. The % lifetime can be calculated as described in Example 4. In some embodiments, the variant narrow channel alpha hemolysin nanopores have a % lifetime of greater than 60%. In some embodiments, the variant narrow channel alpha hemolysin nanopores have a % lifetime of greater than 70%. In some embodiments, the variant narrow channel alpha hemolysin nanopores have a % lifetime of greater than 75%. In some embodiments, the variant narrow channel alpha hemolysin nanopores have a % lifetime of greater than 80%.

In some of the embodiments described herein, the variant narrow channel alpha hemolysin nanopores are characterized according to their “arrival rate.” In this context, the “arrival rate” shall mean the mean arrival rate of a T40-tagged Streptavidin on a 6:1 narrow channel alpha hemolysin nanopore during a 15 minute exposure to a 50 Hz, 150 mV waveform, wherein the “6” component is the variant narrow channel alpha hemolysin subunit and the “1” component” is subunit G2043. The arrival rate can be calculated as described in Example 4. In some embodiments, the variant narrow channel alpha hemolysin nanopores have an arrival rate of less than 25 ms. In some embodiments, the variant narrow channel alpha hemolysin nanopores have an arrival rate of less than 20 ms. In some embodiments, the variant narrow channel alpha hemolysin nanopores have an arrival rate of less than 15 ms.

In certain exemplary embodiments, the variant narrow channel alpha hemolysin subunits provided herein have 80%, 85%, 90%, 95% or more identity to the sequence set forth as SEQ ID NO:1, with the proviso that said amino acid sequence comprises (a) either or both of a D127G substitution relative to SEQ ID NO: 1 and a D128K substitution, and further comprises (b) an amino acid at either or both of E111 and K147 relative to SEQ ID NO: 1 with a side chain that is longer than asparagine, such as glutamic acid, lysine, arginine, or glutamine, and/or (c) an amino acid at M113 relative to SEQ ID NO: 1 with a side chain that is longer than alanine, such as leucine, isoleucine, valine, or methionine. The amino acids at E111, K147, and/or M113 are selected such that the variant narrow channel alpha hemolysin subunit has a threaded rate that is less than the threaded rate of pore P-0304. In an embodiment, the amino acids at E111, K147, and/or M113 are selected such that the variant narrow channel alpha hemolysin subunit has a threaded rate of less than 15%. In another embodiment, the amino acids at E111, K147, and/or M113 are selected such that the variant narrow channel alpha hemolysin subunit has a threaded rate of less than 10%. In another embodiment, the amino acids at E111, K147, and/or M113 are selected such that the variant narrow channel alpha hemolysin subunit has a threaded rate of less than 5%. In another embodiment, the amino acids at E111, K147, and/or M113 are selected such that the variant narrow channel alpha hemolysin subunit has a threaded rate of less than 2%. In yet another embodiment, the variant narrow channel alpha hemolysin subunit comprises each of E111, M113, and K147.

In another embodiment, the variant narrow channel alpha hemolysin subunit comprises an amino acid sequence having at least 75%, 80%, 90%, 95%, 98%, or more identity to SEQ ID NO: 2, wherein the amino acid sequence (a) comprises each of G127 and K128, and further comprises (b) an amino acid at either or both of E111 and K147 relative to SEQ ID NO: 2 with a side chain that is longer than asparagine, such as glutamic acid, lysine, arginine, or glutamine, and/or (c) an amino acid at M113 relative to SEQ ID NO: 2 with a side chain that is longer than alanine, such as leucine, isoleucine, valine, or methionine. The amino acids at E111, K147, and/or M113 are selected such that the variant narrow channel alpha hemolysin subunit has a threaded rate that is less than the threaded rate of pore P-0304. In an embodiment, the amino acids at E111, K147, and/or M113 are selected such that the variant narrow channel alpha hemolysin subunit has a threaded rate of less than 15%. In another embodiment, the amino acids at E111, K147, and/or M113 are selected such that the variant narrow channel alpha hemolysin subunit has a threaded rate of less than 10%. In another embodiment, the amino acids at E111, K147, and/or M113 are selected such that the variant narrow channel alpha hemolysin subunit has a threaded rate of less than 5%. In another embodiment, the amino acids at E111, K147, and/or M113 are selected such that the variant narrow channel alpha hemolysin subunit has a threaded rate of less than 2%. In yet another embodiment, the variant narrow channel alpha hemolysin subunit comprises each of E111, M113, and K147. In yet another embodiment, the variant narrow channel alpha hemolysin subunit comprises or consists of SEQ ID NO: 2.

In another embodiment, the variant narrow channel alpha hemolysin subunit comprises an amino acid sequence having at least 75%, 80%, 90%, 95%, 98%, or more identity to SEQ ID NO: 3, wherein the amino acid sequence (a) comprises each of G127 and K128, and further comprises (b) an amino acid at either or both of E111 and K147 relative to SEQ ID NO: 3 with a side chain that is longer than asparagine, such as glutamic acid, lysine, arginine, or glutamine, and/or (c) an amino acid at M113 relative to SEQ ID NO: 3 with a side chain that is longer than alanine, such as leucine, isoleucine, valine, or methionine. The amino acids at E111, K147, and/or M113 are selected such that the variant narrow channel alpha hemolysin subunit has a threaded rate that is less than the threaded rate of pore P-0304. In an embodiment, the amino acids at E111, K147, and/or M113 are selected such that the variant narrow channel alpha hemolysin subunit has a threaded rate of less than 15%. In another embodiment, the amino acids at E111, K147, and/or M113 are selected such that the variant narrow channel alpha hemolysin subunit has a threaded rate of less than 10%. In another embodiment, the amino acids at E111, K147, and/or M113 are selected such that the variant narrow channel alpha hemolysin subunit has a threaded rate of less than 5%. In another embodiment, the amino acids at E111, K147, and/or M113 are selected such that the variant narrow channel alpha hemolysin subunit has a threaded rate of less than 2%. In yet another embodiment, the variant narrow channel alpha hemolysin subunit comprises each of E111, M113, and K147. In yet another embodiment, the variant narrow channel alpha hemolysin subunit comprises or consists of SEQ ID NO: 3.

In certain example embodiments, the variant narrow channel alpha hemolysin subunit has 75%, 80%, 85%, 90%, 95% or more identity to the sequence set forth as SEQ ID NO: 4, with the proviso that said amino acid sequence comprises (a) each of G127 and K128 of SEQ ID NO: 4, and further comprises (b) an amino acid at either or both of N111 and N147 relative to SEQ ID NO: 4 with a side chain that is longer than asparagine, such as glutamic acid, lysine, arginine, or glutamine, and/or (c) an amino acid at A113 relative to SEQ ID NO: 4 with a side chain that is longer than alanine, such as leucine, isoleucine, valine, or methionine. The amino acids at N111, N147, and/or A113 are selected such that the variant narrow channel alpha hemolysin subunit has a threaded rate that is less than the threaded rate of pore P-0304. In an embodiment, the amino acids at N111, N147, and/or A113 are selected such that the variant narrow channel alpha hemolysin subunit has a threaded rate of less than 15%. In another embodiment, the amino acids at N111, N147, and/or A113 are selected such that the variant narrow channel alpha hemolysin subunit has a threaded rate of less than 10%. In another embodiment, the amino acids at N111, N147, and/or A113 are selected such that the variant narrow channel alpha hemolysin subunit has a threaded rate of less than 5%. In another embodiment, the amino acids at N111, N147, and/or A113 are selected such that the variant narrow channel alpha hemolysin subunit has a threaded rate of less than 2%. In yet another embodiment, the variant narrow channel alpha hemolysin subunit comprises each of N111E, A113M, and N147K substitutions relative to SEQ ID NO: 4. In another embodiment, the amino acids at N111, N147, and/or A113 are selected such that the variant narrow channel alpha hemolysin subunit has a threaded rate of less than 2%. In yet another embodiment, the variant narrow channel alpha hemolysin subunit comprises each of N111E, A113M, and N147K substitutions relative to SEQ ID NO: 4.

In certain example embodiments, the variant narrow channel alpha hemolysin subunit has 75%, 80%, 85%, 90%, 95% or more identity to the sequence set forth as SEQ ID NO: 5, with the proviso that said amino acid sequence comprises: (a) either or both of (a1) G127 of SEQ ID NO: 5, and (a2) a G128K substitution relative to SEQ ID NO: 5, and further comprises (b) an amino acid at either or both of N111 and N147 relative to SEQ ID NO: 5 with a side chain that is longer than asparagine, such as glutamic acid, lysine, arginine, or glutamine, and/or (c) an amino acid at A113 relative to SEQ ID NO: 5 with a side chain that is longer than alanine, such as leucine, isoleucine, valine, or methionine. The amino acids at N111, N147, and/or A113 are selected such that the variant narrow channel alpha hemolysin subunit has a threaded rate that is less than the threaded rate of pore P-0304. In an embodiment, the amino acids at N111, N147, and/or A113 are selected such that the variant narrow channel alpha hemolysin subunit has a threaded rate of less than 15%. In another embodiment, the amino acids at N111, N147, and/or A113 are selected such that the variant narrow channel alpha hemolysin subunit has a threaded rate of less than 10%. In another embodiment, the amino acids at N111, N147, and/or A113 are selected such that the variant narrow channel alpha hemolysin subunit has a threaded rate of less than 5%. In another embodiment, the amino acids at N111, N147, and/or A113 are selected such that the variant narrow channel alpha hemolysin subunit has a threaded rate of less than 2%. In yet another embodiment, the variant narrow channel alpha hemolysin subunit comprises each of N111E, A113M, and N147K substitutions relative to SEQ ID NO: 5. In another embodiment, the amino acids at N111, N147, and/or A113 are selected such that the variant narrow channel alpha hemolysin subunit has a threaded rate of less than 2%. In yet another embodiment, the variant narrow channel alpha hemolysin subunit comprises each of N111E, A113M, and N147K substitutions relative to SEQ ID NO: 5.

In certain example embodiments, the variant narrow channel alpha hemolysin subunit has 75%, 80%, 85%, 90%, 95% or more identity to the sequence set forth as

SEQ ID NO: 6, with the proviso that said amino acid sequence comprises: (a) either or both of a D127G and a D128K substitution relative to SEQ ID NO: 6, and (b) an amino acid at either or both of E111 and K147 relative to SEQ ID NO: 6 with a side chain that is longer than asparagine, such as glutamic acid, lysine, arginine, or glutamine, and/or (c) an amino acid at M113 relative to SEQ ID NO: 6 with a side chain that is longer than alanine, such as leucine, isoleucine, valine, or methionine. The amino acids at E111, K147, and/or M113 are selected such that the variant narrow channel alpha hemolysin subunit has a threaded rate that is less than the threaded rate of pore P-0304. In an embodiment, the amino acids at E111, K147, and/or M113 are selected such that the variant narrow channel alpha hemolysin subunit has a threaded rate of less than 15%. In another embodiment, the amino acids at E111, K147, and/or M113 are selected such that the variant narrow channel alpha hemolysin subunit has a threaded rate of less than 10%. In another embodiment, the amino acids at E111, K147, and/or M113 are selected such that the variant narrow channel alpha hemolysin subunit has a threaded rate of less than 5%. In another embodiment, the amino acids at E111, K147, and/or M113 are selected such that the variant narrow channel alpha hemolysin subunit has a threaded rate of less than 2%. In another embodiment, the amino acids at E111, K147, and/or M113 are selected such that the variant narrow channel alpha hemolysin subunit has a threaded rate of less than 2%. In yet another embodiment, the variant narrow channel alpha hemolysin subunit comprises each of E111, K147, and M113 relative to SEQ ID NO: 6.

In certain example embodiments, the variant narrow channel alpha hemolysin subunit has 75%, 80%, 85%, 90%, 95% or more identity to the sequence set forth as

SEQ ID NO: 7, with the proviso that said amino acid sequence comprises: (a) either or both of a D127G and a D128K substitution relative to SEQ ID NO: 7, and (b) an amino acid at either or both of E111 and K147 relative to SEQ ID NO: 7 with a side chain that is longer than asparagine, such as glutamic acid, lysine, arginine, or glutamine, and/or (c) an amino acid at M113 relative to SEQ ID NO: 7 with a side chain that is longer than alanine, such as leucine, isoleucine, valine, or methionine. The amino acids at E111, K147, and/or M113 are selected such that the variant narrow channel alpha hemolysin subunit has a threaded rate that is less than the threaded rate of pore P-0304. In an embodiment, the amino acids at E111, K147 and/or M113 are selected such that the variant narrow channel alpha hemolysin subunit has a threaded rate of less than 15%. In another embodiment, the amino acids at E111, K147, and/or M113 are selected such that the variant narrow channel alpha hemolysin subunit has a threaded rate of less than 10%. In another embodiment, the amino acids at E111, K147, and/or M113 are selected such that the variant narrow channel alpha hemolysin subunit has a threaded rate of less than 5%. In another embodiment, the amino acids at E111, K147, and/or M113 are selected such that the variant narrow channel alpha hemolysin subunit has a threaded rate of less than 2%. In another embodiment, the amino acids at E111, K147, and/or M113 are selected such that the variant narrow channel alpha hemolysin subunit has a threaded rate of less than 2%. In yet another embodiment, the variant narrow channel alpha hemolysin subunit comprises each of E111, K147, and M113 relative to SEQ ID NO: 7.

In certain example embodiments, the variant narrow channel alpha hemolysin subunit has 75%, 80%, 85%, 90%, 95% or more identity to the sequence set forth as

SEQ ID NO: 8, with the proviso that said amino acid sequence comprises: (a) either or both of a D127G and a D128K substitution relative to SEQ ID NO: 8, and (b) an amino acid at either or both of E111 and K147 relative to SEQ ID NO: 8 with a side chain that is longer than asparagine, such as glutamic acid, lysine, arginine, or glutamine, and/or (c) an amino acid at M113 relative to SEQ ID NO: 8 with a side chain that is longer than alanine, such as leucine, isoleucine, valine, or methionine. The amino acids at E111, K147, and/or M113 are selected such that the variant narrow channel alpha hemolysin subunit has a threaded rate that is less than the threaded rate of pore P-0304. In an embodiment, the amino acids at E111, K147, and/or M113 are selected such that the variant narrow channel alpha hemolysin subunit has a threaded rate of less than 15%. In another embodiment, the amino acids at E111, K147, and/or M113 are selected such that the variant narrow channel alpha hemolysin subunit has a threaded rate of less than 10%. In another embodiment, the amino acids at E111, K147, and/or M113 are selected such that the variant narrow channel alpha hemolysin subunit has a threaded rate of less than 5%. In another embodiment, the amino acids at E111, K147, and/or M113 are selected such that the variant narrow channel alpha hemolysin subunit has a threaded rate of less than 2%. In another embodiment, the amino acids at E111, K147, and/or M113 are selected such that the variant narrow channel alpha hemolysin subunit has a threaded rate of less than 2%. In yet another embodiment, the variant narrow channel alpha hemolysin subunit comprises each of E111, K147, and M113 relative to SEQ ID NO: 8.

The variant narrow channel alpha hemolysin subunits disclosed herein may contain further modifications relative to any of SEQ ID NO: 1-8 that alter or improve characteristics of the resulting nanopores. Numerous schemes and mutations for generating alpha-hemolysin variants useful for nanopore-based sequencing have been described in the art, including, for example, at Noskov, Bhattacharya, Stoddart, PCT/US2015/57902, U.S. Pat. No. 10,301,31, PCT/EP2016/072220, U.S. Pat. No. 10,227,645, PCT/US2017/028636, U.S. Pat. No. 10,351,908, PCT/EP2017/065972, U.S. Pat. No. 10,934,582, PCT/EP2019/054792, US 2020-0385433, each of which is incorporated herein by reference. As one non-limiting example, the present variant narrow channel alpha hemolysin subunits may include a substitution that controls the ability of non-oligomerized alpha hemolysin subunits to self-oligomerize. For example, alpha hemolysin subunits having substitutions at H35 (e.g., H35G/L/D/E substitutions) are substantially non-oligomerized as long as they are kept at room temperature or below (e.g. 25° C. or lower), but will stably oligomerize when the temperature is raised to a higher temperature (e.g. 35° C.). Other examples of substitution strategies for controlling self-oligomerization and/or directing specific patterns of oligomerization are disclosed at, for example, WO 2017-050718. Another example includes substitutions that reduce coefficient of variation of the arrival rate of the pore (CV), such as D227N. In some embodiments, the variant narrow channel alpha hemolysin subunit has a set of modifications relative to any of SEQ ID NO: 1-8 that results in a lifetime of ≥80%. In some embodiments, the variant narrow channel alpha hemolysin subunit has a set of modifications relative to any of SEQ ID NO: 1-8 that results in an arrival rate of ≤15 ms. In some embodiments, the variant narrow channel alpha hemolysin subunit has a set of modifications relative to any of SEQ ID NO: 1-8 that results in a lifetime of ≥80% and an arrival rate of ≤15 ms. In yet other embodiments, the variant narrow channel alpha hemolysin subunit has a set of modifications relative to any of SEQ ID NO: 1-8 that results in a lifetime of ≥80%, an arrival rate of ≤15 ms, and a threaded rate of less than 2%.

The polypeptides may comprise from 1 to 7 variant narrow channel alpha hemolysin subunits. In an embodiment, the polypeptides disclosed herein comprise a single α variant narrow channel alpha hemolysin subunit. In another embodiment, the polypeptide is a concatenated alpha hemolysin polypeptide, comprising from 2 to 7 variant narrow channel alpha hemolysin subunits, explicitly including polypeptides comprising 2 narrow channel alpha hemolysin subunits, polypeptides comprising narrow channel alpha hemolysin subunits, polypeptides comprising 4 narrow channel alpha hemolysin subunits, polypeptides comprising 5 narrow channel alpha hemolysin subunits, polypeptides comprising 6 narrow channel alpha hemolysin subunits, and polypeptides comprising 7 narrow channel alpha hemolysin subunits. Exemplary methods of generating concatenated alpha hemolysin polypeptide and considerations for doing so are disclosed by, for example, Hammerstein and US 2017-0088890 A1. In an embodiment, each narrow channel alpha hemolysin subunit of the concatenated narrow channel alpha hemolysin polypeptide is separated from the other narrow channel alpha hemolysin subunit(s) by a linker sequence. In an embodiment, the linker sequence is a flexible linker. Exemplary flexible linkers are disclosed by, for example, Hammerstein and Chen.

The polypeptides may also include components useful for purification of the polypeptide, such as, for example, epitope tags, protease cleavage sites, etc.

The polypeptides may also include entities useful for attachment of other active agents (such as polymerases) to the polypeptide (referred to herein as “attachment components”). Exemplary attachment components include, for example, components of the Spy Tag/SpyCatcher peptide system (Zakeri et al. PNAS 109: E690-E697 2012), native chemical ligation system (Thapa et al., Molecules 19:14461-14483 2014), sortase system (Wu and Guo, J Carbohydr Chem 31:48-66 2012; Heck et al., Appl Microbiol Biotechnol 97:461-475 2013)), transglutaminase systems (Dennler et al., Bioconjug Chem 25:569 578 2014), formylglycine linkage systems (Rashidian et al., Bio conjug Chem 24:1277-1294 2013), a Click chemistry attachment system, or other chemical ligation techniques known in the art.

### V. Nucleic Acids, Expression Cassettes, Expression Vectors, Recombinant Cells, and Methods of Producing Polypeptides

In another aspect of the present disclosure, isolated polynucleotides are provided, said isolated polynucleotide comprising a nucleotide sequence encoding the isolated polypeptides as described in section IV. In an embodiment, the nucleic acid is an expression cassette comprising the nucleotide sequence encoding the polypeptide linked to a set of nucleic acid transcription elements (such as promoters, enhancers, start and stop codons, ribosomal binding sites, and the like) sufficient for transcription of the nucleotide sequence encoding the polypeptide in a prokaryotic or eukaryotic cell or in a cell-free expression system.

In another aspect, a vector is provided comprising the nucleotide encoding the polypeptide. The vectors may, for example, be cloning or expression vectors. Suitable vector backbones include, for example, those routinely used in the art such as plasmids, artificial chromosomes, BACs, or PACs. Numerous vectors and expression systems are commercially available from such corporations as Novagen (Madison, Wis.), Clonetech (Pal Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, et cetera.

In another embodiment, a host cell comprising the expression vector is provided. For example, a host cell useful for production of polypeptides is transformed or transiently or stably transfected with the expression vector. In another aspect of the present disclosure, a method of preparing a variant alpha-hemolysin polypeptide as described herein is provided, the method comprising (a) culturing a host cell comprising an expression vector as disclosed herein under conditions sufficient to induce expression of the polypeptide, and (b) purifying the polypeptide from the host cell. Such methods are well known in the art, and many systems for doing so are commercially available.

### VI. Variant Narrow Channel Alpha Hemolysin Nanopores

In an embodiment, a variant narrow channel alpha hemolysin nanopore or a hybrid nanopore comprising the variant narrow channel alpha hemolysin nanopore as the biological component is provided, the variant narrow channel alpha hemolysin nanopore having the following properties: (a) a lower threaded rate than nanopore P-0304; and (b) increased lifetime relative to nanopore P-0031 (see Table 2).

TABLE 2

| | | | Mutations relative |
|---|---|---|---|
| | Copy # | Subunit ID | to SEQ ID NO: 1 |
| Pore P-0304 | 6 | G1742 | H35G + N47K + E111N + |
| | | (SEQ ID NO: 4) | M113A + D127G + D128K + |
| | | | T129G + K131G + H144A + |
| | | | K147N + V149K |
| | 1 | G1678 | H35G + E111N + M113A + |
| | | (SEQ ID NO: 5) | K147N + D127G + D128G + |
| | | | T129G + K131G |
| Pore P-0031 | 6 | G639 | H35G + H144A + V149K + |
| | | (SEQ ID NO: 6) | N47K |
| | 1 | G1032 | K8D |
| | | (SEQ ID NO: 7) | |

In some embodiments, the variant narrow channel alpha hemolysin nanopore further has an arrival rate that is comparable to or better than the arrival rate of Pore P-0411 or P-0414:

TABLE 3
Pore P-0411

| Copy # | Subunit ID | Mutations relative to SEQ ID NO: 1 |
|---|---|---|
| 6 | G2055 | D13A + H35G + D127G + |
| | (SEQ ID NO: 2) | D128K + H144A + V149K |
| 1 | G2043 | D128K + V149K |
| | (SEQ ID NO: 8) | |

TABLE 4
Pore P-0414

| Copy # | Subunit ID | Mutations relative to SEQ ID NO: 1 |
|---|---|---|
| 6 | G2097 | H35G + N47K + D127G + |
| | (SEQ ID NO: 3) | D128K + H144A + V149K |
| 1 | G2043 | D128K + V149K |
| | (SEQ ID NO: 8) | |

Each subunit of the variant narrow channel alpha hemolysin nanopore may be identical (termed a “homoheptamer”), or at least one subunit of the heptamer may have a modification relative to the others, such as a different primary amino acid sequence and/or a modification to facilitate attachment of a polypeptide (termed a “heteroheptamer”). Heteroheptameric alpha hemolysin nanopores may be referred to herein by a ratio of the species of different subunits used in the nanopore. For example, a “6:1 alpha hemolysin nanopore” has 6 identical subunits and 1 subunit that is different. In such an example, reference to the “6” component shall mean each of the 6 identical subunits, while reference to the “1” component shall mean the 1 different subunit. In some embodiments, each subunit of the alpha hemolysin nanopore is disposed in a polypeptide that does not contain additional subunits (termed herein a “non-oligomerized subunit”). Exemplary methods of making homoheptamers and heteroheptamers from non-oligomerized alpha hemolysin subunits are disclosed at US 2017-0088890 A1. For example, 6:1 heteroheptamers can be generated by mixing two different subunit preparations (for example, one in which the subunit is modified with an entity that can be used to bind to a polymerase and another entity that does not contain such a modification). The entity that is intended to be in excess in the resulting heptamer is provided in a molar excess relative to the other heptamer in the presence of a membrane and the mixture is incubated in an aqueous solution (such as 20 mM Tris-HCl pH 8.0, 200 mM NaCl or 20 mM Sodium Citrate pH 3, 400 mM NaCl, 0.1% TWEEN20+0.2 M TMAO) overnight at 37° C. The resulting heptamers are then purified by cation exchange chromatography. In some embodiments, oligomerization is performed in the presence of trimethylamine N-oxide (TMAO), such as from 0.1 to 5M TMAO, from 1 to 4M TMAO, and the like. In other embodiments, the nanopore includes at least one set of concatenated subunits. Exemplary methods of making alpha hemolysin nanopores from concatenated alpha hemolysin subunits are disclosed at, for example, Hammerstein and US 2017-0088890 A1.

The variant narrow channel alpha hemolysin nanopores described herein may also include a polymerase attached thereto. In an embodiment, a single polymerase is attached to the variant narrow channel alpha hemolysin nanopore. Exemplary polymerases include those derived from DNA polymerase Clostridium phage phiCPV4 (described by GenBank Accession No. YP_00648862, referred to herein as “Pol6”), phi29 DNA polymerase, T7 DNA pol, T4 DNA pol, E. coli DNA pol 1, Klenow fragment, T7 RNA polymerase, and E. coli RNA polymerase, as well as associated subunits and cofactors. In an embodiment, the polymerase is a DNA polymerase derived from Pol6. Exemplary Pol6 derivatives useful in nanopore-based sequencing are disclosed at, for example, US 2016/0222363, US 2016/0333327, US 2017/0267983, US 2018/0094249, and US 2018/0245147. Exemplary methods of attaching a polymerase to an alpha hemolysin nanopore include SpyTag/SpyCatcher peptide system (Zakeri et al. PNAS 109: E690-E697 2012), native chemical ligation system (Thapa et al., Molecules 19:14461-14483 2014), sortase system (Wu and Guo, J Carbohydr Chem 31:48-66 2012; Heck et al., Appl Microbiol Biotechnol 97:461-475 2013)), transglutaminase systems (Dennler et al., Bioconjug Chem 25:569 578 2014), formylglycine linkage systems (Rashidian et al., Bio conjug Chem 24:1277-1294 2013), Click chemistry attachment systems, or other chemical ligation techniques known in the art. In an embodiment, the polymerase is attached to an amino acid side chain of one of the alpha hemolysin subunits. In an embodiment, the alpha hemolysin nanopore is a 6:1 nanopore, wherein the polymerase is attached to the “1” component. In an embodiment, the alpha hemolysin nanopore is a 6:1 nanopore, wherein the polymerase is attached to the “1” component, and wherein the polymerase is a DNA polymerase. In another embodiment, the alpha hemolysin nanopore is a 6:1 nanopore, wherein the polymerase is attached to the “1” component, and wherein the polymerase is a DNA polymerase derived from Pol6.

In some of the embodiments described herein, the variant narrow channel alpha hemolysin nanopores are characterized according to their “threaded rate.” In this context, the “threaded rate” shall mean the percentage of the variant narrow channel alpha hemolysin nanopores with high quality reads (HQRs) that exhibit a threaded state. The percentage of pores with the threaded state can be calculated as described in Example 5. In some embodiments, the variant narrow channel alpha hemolysin nanopores have a threaded rate of less than 15%. In some embodiments, the variant narrow channel alpha hemolysin nanopores have a threaded rate of less than 10%. In some embodiments, the variant narrow channel alpha hemolysin nanopores have a threaded rate of less than 5%. In some embodiments, the variant narrow channel alpha hemolysin nanopores have a threaded rate of less than 2%.

In some of the embodiments described herein, the variant narrow channel alpha hemolysin nanopores are characterized according to their “% lifetime.” In this context, the “% lifetime” shall mean the percentage of the variant narrow channel alpha hemolysin nanopores that remain active to a T40-tagged Streptavidin after 1 hour exposure to a 350 mV sequencing waveform. The % lifetime can be calculated as described in Example 4. In some embodiments, the variant narrow channel alpha hemolysin nanopores have a % lifetime of greater than 60%. In some embodiments, the variant narrow channel alpha hemolysin nanopores have a % lifetime of greater than 70%. In some embodiments, the variant narrow channel alpha hemolysin nanopores have a % lifetime of greater than 75%. In some embodiments, the variant narrow channel alpha hemolysin nanopores have a % lifetime of greater than 80%.

In some of the embodiments described herein, the variant narrow channel alpha hemolysin nanopores are characterized according to their “arrival rate.” In this context, the “arrival rate” shall mean the mean arrival rate of a T40-tagged Streptavidin on the variant narrow channel alpha hemolysin nanopore during a 15 minute exposure to a 50 Hz, 150 mV waveform. The arrival rate can be calculated as described in Example 4. In some embodiments, the variant narrow channel alpha hemolysin nanopores have an arrival rate of less than 25 ms. In some embodiments, the variant narrow channel alpha hemolysin nanopores have an arrival rate of less than 20 ms. In some embodiments, the variant narrow channel alpha hemolysin nanopores have an arrival rate of less than 15 ms.

In an embodiment, the variant narrow channel alpha hemolysin nanopore comprises 1, 2, 3, 4, 5, 6, or 7 narrow channel alpha hemolysin subunits having the following characteristics: (a) at least 75% identity, at least 80% identity, at least 85% identity, at least 90% identity, at least 91% identity, at least 92% identity, at least 93% identity, at least 94% identity, or at least 95% identity with SEQ ID NO: 1; (b) a D127G substitution relative to SEQ ID NO: 1; (c) a D128K substitution relative to SEQ ID NO: 1, and (d) one or more of (dl) an amino acid at either or both of E111 and K147 relative to SEQ ID NO: 1 with a side chain that is longer than asparagine, such as glutamic acid, lysine, arginine, or glutamine, and/or (d2) an amino acid at M113 relative to SEQ ID NO: 1 with a side chain that is longer than alanine, such as leucine, isoleucine, valine, or methionine. The amino acids at E111, K147, and/or M113 are selected such the variant narrow channel alpha hemolysin nanopore has a threaded rate that is less than a threaded rate of pore P-0304. In an embodiment, the variant narrow channel alpha hemolysin nanopore has a threaded rate that is less than 15%. In another embodiment, the variant narrow channel alpha hemolysin nanopore has a threaded rate that is less than 10%. In another embodiment, the variant narrow channel alpha hemolysin nanopore has a threaded rate that is less than 5%. In another embodiment, the variant narrow channel alpha hemolysin nanopore has a threaded rate that is less than 2%. In yet another embodiment, the variant narrow channel alpha hemolysin nanopore is a 6:1 heteroheptamer, wherein: (a) at least the “6” component comprises (a1) either or both of a D127G substitution and a D128K substitution relative to SEQ ID NO: 1, and further comprises (a2) an amino acid at either or both of E111 and K147 relative to SEQ ID NO: 1 with a side chain that is longer than asparagine, such as glutamic acid, lysine, arginine, or glutamine, and/or (a3) an amino acid at M113 relative to SEQ ID NO: 1 with a side chain that is longer than alanine, such as leucine, isoleucine, valine, or methionine; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 1 and further is attached to or adapted to be attached to a polymerase. In another embodiment, the variant narrow channel alpha hemolysin nanopore is a 6:1 heteroheptamer, wherein: (a) at least the “6” component comprises an amino acid sequence having (a1) a D127G substitution relative to SEQ ID NO: 1, (a2) a D128K substitution relative to SEQ ID NO: 1, and (a3) each of E111, M113, and K147 of SEQ ID NO: 1; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 1 and further is attached to or adapted to be attached to a polymerase.

In an embodiment, a variant narrow channel alpha hemolysin nanopore comprises 1, 2, 3, 4, 5, 6, or 7 narrow channel alpha hemolysin subunits having the following characteristics: (a) at least 75% identity, at least 80% identity, at least 85% identity, at least 90% identity, at least 91% identity, at least 92% identity, at least 93% identity, at least 94% identity, or at least 95% identity with SEQ ID NO: 2, (b) comprises each of G127 and K128 of SEQ ID NO: 2, and (c) further comprises (c1) an amino acid at either or both of E111 and K147 relative to SEQ ID NO: 2 with a side chain that is longer than asparagine, such as glutamic acid, lysine, arginine, or glutamine, and/or (c2) an amino acid at M113 relative to SEQ ID NO: 2 with a side chain that is longer than alanine, such as leucine, isoleucine, valine, or methionine. The amino acids at E111, K147, and/or M113 are selected such that the variant narrow channel alpha hemolysin has a threaded rate that is less than the threaded rate of pore P-0304. In an embodiment, the amino acids at E111, K147, and/or M113 are selected such that the variant narrow channel alpha hemolysin nanopore has a threaded rate of less than 15%. In another embodiment, the amino acids at E111, K147, and/or M113 are selected such that the variant narrow channel alpha hemolysin nanopore has a threaded rate of less than 10%. In another embodiment, the amino acids at E111, K147, and/or M113 are selected such that the variant narrow channel alpha hemolysin nanopore has a threaded rate of less than 5%. In another embodiment, the amino acids at E111, K147, and/or M113 are selected such that the variant narrow channel alpha hemolysin nanopore has a threaded rate of less than 2%. In yet another embodiment, the narrow channel alpha hemolysin subunit(s) comprise each of E111, M113, and K147. In yet another embodiment, the narrow channel alpha hemolysin subunit(s) comprise or consist of SEQ ID NO: 2. In yet another embodiment, the variant narrow channel alpha hemolysin nanopore is a 6:1 heteroheptamer, wherein: (a) at least the “6” component (a1) comprises each of G127 and K128 relative to SEQ ID NO: 2, and further comprises (a2) an amino acid at either or both of E111 and K147 relative to SEQ ID NO: 2 with a side chain that is longer than asparagine, such as glutamic acid, lysine, arginine, or glutamine, and/or (a3) an amino acid at M113 relative to SEQ ID NO: 2 with a side chain that is longer than alanine, such as leucine, isoleucine, valine, or methionine; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 2 and further is attached to or adapted to be attached to a polymerase. In another embodiment, the variant narrow channel alpha hemolysin nanopore is a 6:1 heteroheptamer, wherein: (a) at least the “6” component comprises each of G127, K128, E111, M113, and K147 of SEQ ID NO: 2; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 2 and further is attached to or adapted to be attached to a polymerase.

In an embodiment, a variant narrow channel alpha hemolysin nanopore comprises 1, 2, 3, 4, 5, 6, or 7 narrow channel alpha hemolysin subunits having the following characteristics: (a) at least 75% identity, at least 80% identity, at least 85% identity, at least 90% identity, at least 91% identity, at least 92% identity, at least 93% identity, at least 94% identity, or at least 95% identity with SEQ ID NO: 3, (b) comprises each of G127 and K128 of SEQ ID NO: 3, and (c) further comprises (c1) an amino acid at either or both of E111 and K147 relative to SEQ ID NO: 3 with a side chain that is longer than asparagine, such as glutamic acid, lysine, arginine, or glutamine, and/or (c2) an amino acid at M113 relative to SEQ ID NO: 3 with a side chain that is longer than alanine, such as leucine, isoleucine, valine, or methionine. The amino acids at E111, K147, and/or M113 are selected such that the variant narrow channel alpha hemolysin has a threaded rate that is less than the threaded rate of pore P-0304. In an embodiment, the amino acids at E111, K147, and/or M113 are selected such that the variant narrow channel alpha hemolysin nanopore has a threaded rate of less than 15%. In another embodiment, the amino acids at E111, K147, and/or M113 are selected such that the variant narrow channel alpha hemolysin nanopore has a threaded rate of less than 10%. In another embodiment, the amino acids at E111, K147, and/or M113 are selected such that the variant narrow channel alpha hemolysin nanopore has a threaded rate of less than 5%. In another embodiment, the amino acids at E111, K147, and/or M113 are selected such that the variant narrow channel alpha hemolysin nanopore has a threaded rate of less than 2%. In yet another embodiment, the narrow channel alpha hemolysin subunit(s) comprise each of E111, M113, and K147. In yet another embodiment, the narrow channel alpha hemolysin subunit(s) comprise or consist of SEQ ID NO: 3. In yet another embodiment, the variant narrow channel alpha hemolysin nanopore is a 6:1 heteroheptamer, wherein: (a) at least the “6” component (a1) comprises each of G127 and K128 relative to SEQ ID NO: 3, and further comprises (a2) an amino acid at either or both of E111 and K147 relative to SEQ ID NO: 3 with a side chain that is longer than asparagine, such as glutamic acid, lysine, arginine, or glutamine, and/or (a3) an amino acid at M113 relative to SEQ ID NO: 3 with a side chain that is longer than alanine, such as leucine, isoleucine, valine, or methionine; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 3 and further is attached to or adapted to be attached to a polymerase. In another embodiment, the variant narrow channel alpha hemolysin nanopore is a 6:1 heteroheptamer, wherein: (a) at least the “6” component comprises each of G127, K128, E111, M113, and K147 of SEQ ID NO: 3; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 3 and further is attached to or adapted to be attached to a polymerase.

In an embodiment, a variant narrow channel alpha hemolysin nanopore comprises 1, 2, 3, 4, 5, 6, or 7 narrow channel alpha hemolysin subunits having the following characteristics: (a) at least 75% identity, at least 80% identity, at least 85% identity, at least 90% identity, at least 91% identity, at least 92% identity, at least 93% identity, at least 94% identity, or at least 95% identity with SEQ ID NO: 4, (b) each of G127 and K128 of SEQ ID NO: 4, and (c) further comprises (c1) an amino acid at either or both of N111 and N147 relative to SEQ ID NO: 4 with a side chain that is longer than asparagine, such as glutamic acid, lysine, arginine, or glutamine, and/or (c2) an amino acid at A113 relative to SEQ ID NO: 4 with a side chain that is longer than alanine, such as leucine, isoleucine, valine, or methionine. The amino acids at N111, N147, and/or A113 are selected such that the variant narrow channel alpha hemolysin has a threaded rate that is less than the threaded rate of pore P-0304. In an embodiment, the amino acids at N111, N147, and/or A113 are selected such that the variant narrow channel alpha hemolysin nanopore has a threaded rate of less than 15%. In another embodiment, the amino acids at N111, N147, and/or A113 are selected such that the variant narrow channel alpha hemolysin nanopore has a threaded rate of less than 10%. In another embodiment, the amino acids at N111, N147, and/or A113 are selected such that the variant narrow channel alpha hemolysin nanopore has a threaded rate of less than 5%. In another embodiment, the amino acids at N111, N147, and/or A113 are selected such that the variant narrow channel alpha hemolysin nanopore has a threaded rate of less than 2%. In yet another embodiment, the polypeptide comprises each of N111E, A113M, and N147K substitutions relative to SEQ ID NO: 4. In yet another embodiment, the polypeptide comprises each of G127 and K128 relative to SEQ ID NO: 4 and further comprises each of N111E, A113M, and N147K substitutions relative to SEQ ID NO: 4. In yet another embodiment, the variant narrow channel alpha hemolysin nanopore is a 6:1 heteroheptamer, wherein: (a) at least the “6” component (al) comprises each of G127 and K128 relative to SEQ ID NO: 4, and further comprises (a2) an amino acid at either or both of N111 and N147 relative to SEQ ID NO: 4 with a side chain that is longer than asparagine, such as glutamic acid, lysine, arginine, or glutamine, and/or (a3) an amino acid at A113 relative to SEQ ID NO: 4 with a side chain that is longer than alanine, such as leucine, isoleucine, valine, or methionine; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 4 and further is attached to or adapted to be attached to a polymerase. In yet another embodiment, the variant narrow channel alpha hemolysin nanopore is a 6:1 heteroheptamer, wherein: (a) at least the “6” component comprises each of G127 and K128 relative to SEQ ID NO: 4, and further comprises each of N111E, N147K, A113M substitutions relative to SEQ ID NO: 4; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 4 and further is attached to or adapted to be attached to a polymerase.

In an embodiment, a variant narrow channel alpha hemolysin nanopore comprises 1, 2, 3, 4, 5, 6, or 7 narrow channel alpha hemolysin subunits having the following characteristics: (a) at least 75% identity, at least 80% identity, at least 85% identity, at least 90% identity, at least 91% identity, at least 92% identity, at least 93% identity, at least 94% identity, or at least 95% identity with SEQ ID NO: 5, (b) comprises (b1) G127 of SEQ ID NO: 5, and (b2) a G128K substitution relative to SEQ ID NO: 5, and (c) further comprises (c1) an amino acid at either or both of N111 and N147 relative to SEQ ID NO: 5 with a side chain that is longer than asparagine, such as glutamic acid, lysine, arginine, or glutamine, and/or (c2) an amino acid at A113 relative to SEQ ID NO: 5 with a side chain that is longer than alanine, such as leucine, isoleucine, valine, or methionine. The amino acids at N111, N147, and/or A113 are selected such that the variant narrow channel alpha hemolysin has a threaded rate that is less than the threaded rate of pore P-0304. In an embodiment, the amino acids at N111, N147, and/or A113 are selected such that the variant narrow channel alpha hemolysin nanopore has a threaded rate of less than 15%. In another embodiment, the amino acids at N111, N147, and/or A113 are selected such that the variant narrow channel alpha hemolysin nanopore has a threaded rate of less than 10%. In another embodiment, the amino acids at N111, N147, and/or A113 are selected such that the variant narrow channel alpha hemolysin nanopore has a threaded rate of less than 5%. In another embodiment, the amino acids at N111, N147, and/or A113 are selected such that the variant narrow channel alpha hemolysin nanopore has a threaded rate of less than 2%. In yet another embodiment, the polypeptide comprises each of N111E, A113M, and N147K substitutions relative to SEQ ID NO: 5. In yet another embodiment, the polypeptide comprises G127 of SEQ ID NO: 5 and G128K, N111E, A113M, and N147K substitutions relative to SEQ ID NO: 5. In yet another embodiment, the variant narrow channel alpha hemolysin nanopore is a 6:1 heteroheptamer, wherein: (a) at least the “6” component comprises: (a1) G127 of SEQ ID NO: 5, (a2) a G128K substitution relative to SEQ ID NO: 5, (a3) an amino acid at either or both of N111 and N147 relative to SEQ ID NO: 5 with a side chain that is longer than asparagine, such as glutamic acid, lysine, arginine, or glutamine, and (a4) an amino acid at A113 relative to SEQ ID NO: 5 with a side chain that is longer than alanine, such as leucine, isoleucine, valine, or methionine; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 5 and further is attached to or adapted to be attached to a polymerase. In yet another embodiment, the variant narrow channel alpha hemolysin nanopore is a 6:1 heteroheptamer, wherein: (a) at least the “6” component comprises G127 of SEQ ID NO: 5 and each of G128K, N111E, N147K, A113M substitutions relative to SEQ ID NO: 5; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 5 and further is attached to or adapted to be attached to a polymerase.

In an embodiment, a variant narrow channel alpha hemolysin nanopore comprises 1, 2, 3, 4, 5, 6, or 7 narrow channel alpha hemolysin subunits having the following characteristics: (a) at least 75% identity, at least 80% identity, at least 85% identity, at least 90% identity, at least 91% identity, at least 92% identity, at least 93% identity, at least 94% identity, or at least 95% identity with SEQ ID NO: 6 is provided, (b) either or both of a D127G substitution and a D128K substitution relative to SEQ ID NO: 6, and (c) further comprises (c1) an amino acid at either or both of E111 and K147 relative to SEQ ID NO: 6 with a side chain that is longer than asparagine, such as glutamic acid, lysine, arginine, or glutamine, and/or (c2) an amino acid at M113 relative to SEQ ID NO: 6 with a side chain that is longer than alanine, such as leucine, isoleucine, valine, or methionine. The amino acids at E111, K147, and/or M113 are selected such the percentage of nanopores showing a threaded state is reduced relative to pore P-0304. In an embodiment, the variant narrow channel alpha hemolysin nanopore has a threaded rate that is less than 15%. In another embodiment, the variant narrow channel alpha hemolysin nanopore has a threaded rate that is less than 10%. In another embodiment, the variant narrow channel alpha hemolysin nanopore has a threaded rate that is less than 5%. In another embodiment, the variant narrow channel alpha hemolysin nanopore has a threaded rate that is less than 2%. In yet another embodiment, the variant narrow channel alpha hemolysin nanopore is a 6:1 heteroheptamer, wherein: (a) at least the “6” component comprises (a1) either or both of a D127G substitution and a D128K substitution relative to SEQ ID NO: 6, and further comprises (a2) an amino acid at either or both of E111 and K147 relative to SEQ ID NO: 6 with a side chain that is longer than asparagine, such as glutamic acid, lysine, arginine, or glutamine, and/or (a3) an amino acid at M113 relative to SEQ ID NO: 6 with a side chain that is longer than alanine, such as leucine, isoleucine, valine, or methionine; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 6 and further is attached to or adapted to be attached to a polymerase. In another embodiment, the variant narrow channel alpha hemolysin nanopore is a 6:1 heteroheptamer, wherein: (a) at least the “6” component comprises an amino acid sequence having (a1) a D127G substitution relative to SEQ ID NO: 6, (a2) a D128K substitution relative to SEQ ID NO: 6, and (a3) each of E111, M113, and K147 of SEQ ID NO: 6; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 6 and further is attached to or adapted to be attached to a polymerase.

In an embodiment, a variant narrow channel alpha hemolysin nanopore comprises 1, 2, 3, 4, 5, 6, or 7 narrow channel alpha hemolysin subunits having the following characteristics: (a) at least 75% identity, at least 80% identity, at least 85% identity, at least 90% identity, at least 91% identity, at least 92% identity, at least 93% identity, at least 94% identity, or at least 95% identity with SEQ ID NO: 7, (b) either or both of a D127G substitution and a D128K substitution relative to SEQ ID NO: 7, and (c) further comprises (c1) an amino acid at either or both of E111 and K147 relative to SEQ ID NO: 7 with a side chain that is longer than asparagine, such as glutamic acid, lysine, arginine, or glutamine, and/or (c2) an amino acid at M113 relative to SEQ ID NO: 7 with a side chain that is longer than alanine, such as leucine, isoleucine, valine, or methionine. The amino acids at E111, K147, and/or M113 are selected such the percentage of nanopores showing a threaded state is reduced relative to pore P-0304. In an embodiment, the variant narrow channel alpha hemolysin nanopore has a threaded rate that is less than 15%. In another embodiment, the variant narrow channel alpha hemolysin nanopore has a threaded rate that is less than 10%. In another embodiment, the variant narrow channel alpha hemolysin nanopore has a threaded rate that is less than 5%. In another embodiment, the variant narrow channel alpha hemolysin nanopore has a threaded rate that is less than 2%. In yet another embodiment, the variant narrow channel alpha hemolysin nanopore is a 6:1 heteroheptamer, wherein: (a) at least the “6” component comprises (a1) either or both of a D127G substitution and a D128K substitution relative to SEQ ID NO: 7, and further comprises (a2) an amino acid at either or both of E111 and K147 relative to SEQ ID NO: 7 with a side chain that is longer than asparagine, such as glutamic acid, lysine, arginine, or glutamine, and/or (a3) an amino acid at M113 relative to SEQ ID NO: 7 with a side chain that is longer than alanine, such as leucine, isoleucine, valine, or methionine; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 7 and further is attached to or adapted to be attached to a polymerase. In another embodiment, the variant narrow channel alpha hemolysin nanopore is a 6:1 heteroheptamer, wherein: (a) at least the “6” component comprises an amino acid sequence having (a1) a D127G substitution relative to SEQ ID NO: 7, (a2) a D128K substitution relative to SEQ ID NO: 7, and (a3) each of E111, M113, and K147 of SEQ ID NO: 7; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 7 and further is attached to or adapted to be attached to a polymerase.

In an embodiment, a variant narrow channel alpha hemolysin nanopore comprises 1, 2, 3, 4, 5, 6, or 7 narrow channel alpha hemolysin subunits having the following characteristics: (a) at least 75% identity, at least 80% identity, at least 85% identity, at least 90% identity, at least 91% identity, at least 92% identity, at least 93% identity, at least 94% identity, or at least 95% identity with SEQ ID NO: 8, (b) a D127G substitution and a D128K substitution relative to SEQ ID NO: 8, and (c) further comprises (c1) an amino acid at either or both of E111 and K147 relative to SEQ ID NO: 8 with a side chain that is longer than asparagine, such as glutamic acid, lysine, arginine, or glutamine, and/or (c2) an amino acid at M113 relative to SEQ ID NO: 8 with a side chain that is longer than alanine, such as leucine, isoleucine, valine, or methionine. The amino acids at E111, K147, and/or M113 are selected such the percentage of nanopores showing a threaded state is reduced relative to pore P-0304. In an embodiment, the variant narrow channel alpha hemolysin nanopore has a threaded rate that is less than 15%. In another embodiment, the variant narrow channel alpha hemolysin nanopore has a threaded rate that is less than 10%. In another embodiment, the variant narrow channel alpha hemolysin nanopore has a threaded rate that is less than 5%. In another embodiment, the variant narrow channel alpha hemolysin nanopore has a threaded rate that is less than 2%. In yet another embodiment, the variant narrow channel alpha hemolysin nanopore is a 6:1 heteroheptamer, wherein: (a) at least the “6” component comprises (a1) either or both of a D127G substitution and a D128K substitution relative to SEQ ID NO: 8, and further comprises (a2) an amino acid at either or both of E111 and K147 relative to SEQ ID NO: 8 with a side chain that is longer than asparagine, such as glutamic acid, lysine, arginine, or glutamine, and/or (a3) an amino acid at M113 relative to SEQ ID NO: 8 with a side chain that is longer than alanine, such as leucine, isoleucine, valine, or methionine; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 8 and further is attached to or adapted to be attached to a polymerase. In another embodiment, the variant narrow channel alpha hemolysin nanopore is a 6:1 heteroheptamer, wherein: (a) at least the “6” component comprises an amino acid sequence having (a1) a D127G substitution relative to SEQ ID NO: 8, (a2) a D128K substitution relative to SEQ ID NO: 8, and (a3) each of E111, M113, and K147 of SEQ ID NO: 8; and (b) the “1” component comprises an amino acid sequence having at least 80% identity, at least 85% identity, at least 90% identity, at least 91% identity, at least 92% identity, at least 93% identity, at least 94% identity, or at least 95% identity with SEQ ID NO: 8 and further is attached to or adapted to be attached to a polymerase.

### VII. SBS Sequencing Systems and Methods

In an embodiment, a system for performing nucleic acid sequencing-by-synthesis (SBS) is provided, the system comprising: (a) a variant narrow channel alpha hemolysin nanopore as disclosed in section VI, (b) a nucleic acid polymerase associated with the nanopore, (c) a set of nucleotide oligophosphates disposed in an electrolyte solution, said nucleotide oligophosphates comprising a positively-charged tag capable of threading through the nanopore of (a), and (d) at least one electrode positioned to record a characteristic of a current flowing through the channel.

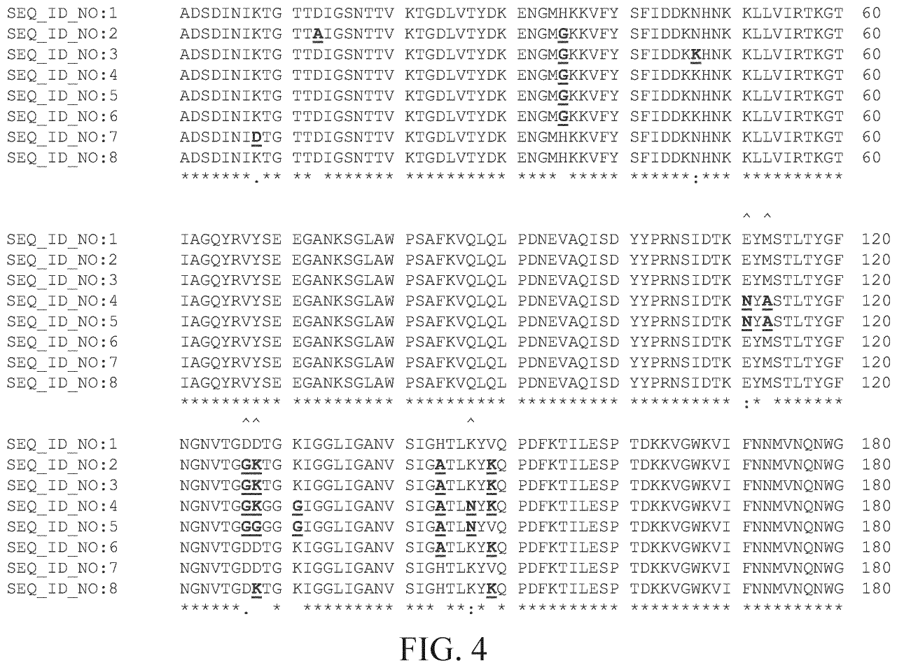

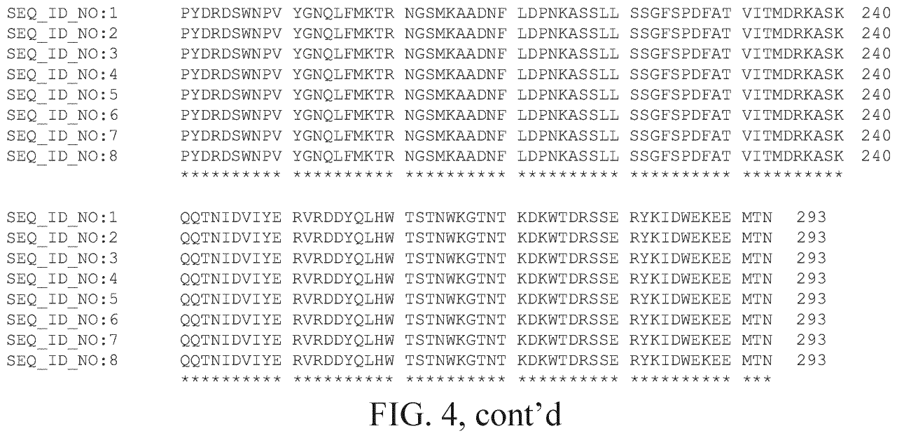

FIG. 4 illustrates an exemplary embodiment of a nanopore sequencing complex 500 for performing a tag-based SBS nucleotide sequencing. An electrically-resistive barrier 501 separates a bulk electrolyte solution 502 from a second electrolyte solution 503. A heptameric alpha hemolysin nanopore as disclosed herein 504 is disposed in the electrically-resistive barrier 501, and the channel of the nanopore 505 provides a path through which ions can flow between the bulk electrolyte 502 and the second electrolyte 503. A working electrode 506 is disposed on the side of the electrically-resistive barrier 501 containing the second electrolyte 503 (termed the “trans side” of the electrically-resistive barrier) and positioned near the heptameric alpha hemolysin nanopore 504. A counter electrode 507 is positioned on the side of the electrically-resistive barrier 501 containing the bulk electrolyte 502 (termed the “cis side” of the electrically-resistive barrier). A signal source 508 is adapted to apply a voltage signal between the working electrode 506 and the counter electrode 507. A polymerase 509 is associated with the heptameric alpha hemolysin nanopore 504, and a primed template nucleic acid 510 is associated with the polymerase. The bulk electrolyte 502 includes four different polymer-tagged nucleoside oligophosphates 511 (tag illustrated as 511a). The polymerase 509 catalyzes incorporation of the polymer-tagged nucleotides 511 into an amplicon of the template. When a polymer-tagged nucleoside oligophosphate 511 is correctly complexed with polymerase 509, the tag 511a can be pulled (e.g., loaded) into the nanopore by an electrical force, such as a force generated in the presence of an electric field generated by a voltage applied across the electrically-resistive barrier 501 and/or nanopore 504. While the tag 511a occupies the channel of the nanopore 504, it affects ionic flow through the nanopore 504, thereby generating an ionic blockade signal 512. Each nucleotide 511 has a unique polymer tag 511a that generates a unique ionic blockade signal due to the distinct chemical structure and/or size of the tag 511a. By identifying the unique ionic blockade signal 512, the identity of the unique tags 511a (and therefore, the nucleotide 510 with which it is associated) can be identified. This process is repeated iteratively with each nucleotide 510 incorporated into the amplicon.

### VIII. EXAMPLES

### Example 1

### Generation and Expression of Variant Alpha-Hemolysin Polypeptides

DNA encoding a wild-type alpha hemolysin having the amino acid sequence of SEQ ID NO: 1 was purchased from a commercial source. Sequence modifications were performed by site-directed mutagenesis using a QuikChange Multi Site-Directed Mutagenesis kit (Agilent, La Jolla, CA) to generate nucleic acids encoding SEQ ID NO: 2-8, with a C-terminal linker/TEV/HisTag. Additionally, each of SEQ ID NO: 5, 7, and 8 were expressed with a C-terminal SpyTag. E.coli BL21 DE3 cells (ThermoFisher, Waltham, MA, USA) were transformed with pET-26b (+) vector and the transformed cells were cultivated for protein expression according to the manufacturer's instructions. The cultivated cells were harvested by centrifugation and then lysed via sonification. Polypeptides bearing the cleavable epitope tag were purified from the lysate by affinity column chromatography (TALON® Metal Affinity Resin, Takara Bio USA). The epitope tags were cleaved and the variant alpha hemolysin polypeptides separated from the cleaved tags and uncleaved polypeptides via affinity column chromatography (TALON® Metal Affinity Resin, Takara Bio USA). The proteins were stored at 4° C. if used within 5 days, otherwise 8% trehalose was added and stored at −80° C. Amino acid sequences of the variant alpha hemolysin polypeptides produced in this manner and their alignment with SEQ ID NO: 1 are illustrated at FIG. 4. The illustrated sequences include on the alpha hemolysin subunit sequences and do not include the associated Spy Tag sequences.

### Example 2

### Assembly of Nanopores

Using approximately 10 mg of total protein, the following alpha hemolysin/SpyTag to desired alpha hemolysin-variant protein combinations were mixed together at a 9:1 ratio (w/w) of subunit 1 to subunit 2 to form a mixture of heptamers:

TABLE 5

| Pore ID | Subunit ID | Substitutions relative to SEQ ID NO: 1 |
|---|---|---|
| P-0304 | Subunit 1: G1742 | H35G + N47K + E111N + M113A + |
| | (SEQ ID NO: 4) | D127G + D128K + T129G + K131G + |
| | | H144A + K147N + V149K |
| | Subunit 2: G1678 | H35G + E111N+ M113A + K147N + |
| | (SEQ ID NO: 5) | D127G + D128G + T129G + K131G |
| P-0031 | Subunit 1: G639 | H35G + H144A + V149K + N47K |
| | (SEQ ID NO: 6) | |
| | Subunit 2: G1032 | K8D |
| | (SEQ ID NO: 7) | |
| P-0411 | Subunit 1: G2055 | D13A + H35G + D127G + D128K + |
| | (SEQ ID NO: 2) | H144A + V149K |
| | Subunit 2: G2043 | D128K + V149K + N293 |
| | (SEQ ID NO: 8) | |
| P-0414 | Subunit 1: G2097 | H35G + N47K + D127G + D128K + |
| | (SEQ ID NO: 3) | H144A + V149K |
| | Subunit 2: G2043 | D128K + V149K + N293 |
| | (SEQ ID NO: 8) | |

Diphytanoylphosphatidylcholine (DPhPC) lipid was solubilized in either 50 mM Tris, 200 mM NaCl, pH 8 or 150 mM KCl, 30 mM HEPES, pH 7.5 to a final concentration of 50 mg/ml and added to the mixture of α-HL subunits to a final concentration of 5 mg/ml. The mixture of the alpha hemolysin subunits was incubated at 37° C. for at least 60 minutes. Thereafter, n-Octyl-β-D-Glucopyranoside (βOG) was added to a final concentration of 5% (weight/volume) to solubilize the resulting lipid-protein mixture. The sample was centrifuged to clear protein aggregates and left over lipid complexes and the supernatant was collected for further purification. The mixture of heptamers was then subjected to cation exchange purification and the elution fraction that corresponded to a 6:1 ratio of subunit 1: subunit 2 was collected.

### Example 3

### Arrival Rate and Lifetime of Pores

To measure the lifetime of the generated nanopores, the 6:1 pores generated in Example 2 are inserted onto a sequencing array as described in in PCT/US14/61853. Streptavidin beads conjugated to a poly-deoxythymidine 40 mer (T40 tag) were flowed onto the array and a sequencing waveform at 350 mV was applied to the system for 1 hour. As the polarity of the charge changed, the tag inserted (resulting in an “inserted state”) and ejected from the pore (resulting in an “open channel”), which was observed by monitoring changes in conductance of each individual pore on the array. Pores were considered to be “active” as long as they continued to display distinct conductance levels correlating to the inserted state and open channel. The “lifetime” of the pore species was determined by calculating the percentage of single pores that remained active throughout the entire 1 hour run.

To measure the arrival rate of the pore, the same setup was used as in the lifetime experiments, except the array was subjected to a 50 Hz, 150 mV waveform for 15 minutes. The “arrival rate” for the pore species was determined by: (a) determining the average time between pore insertions for each individual pore on the array, the (b) calculating the mean of all averages determined in (a).

Each experiment was conducted for all of the pores described in Table 5. Results are reported at FIG. 2, with the lifetime (Y-axis) plotted against the mean arrival rate (X-Axis) for each pore species. As can be seen, the two narrow channel alpha hemolysin nanopores with D127G+D128K substitutions relative to SEQ ID NO: 1 (P-0411 & P-0414) had relatively high lifetimes (>80%) and acceptable arrival rates (<15 ms), comparable to the wide channel alpha hemolysin nanopore (P-0304). The narrow channel alpha hemolysin nanopore without the D127G+D128K substitutions had a much lower lifetime (<10%). This indicates that D127G+D128K substitutions greatly improve the lifetime of narrow channel alpha hemolysin nanopores while preserving acceptable arrival rates.

### Example 5

### Mitigation of Threading Using Narrow Channel Alpha Hemolysin Nanopores

To evaluate the effect of a narrow channel alpha hemolysin nanopore on the extent of template threading, a standard sequencing experiment was run with each of the pores from Example 2.

E.coli BL21 DE3 cells (ThermoFisher, Waltham, MA, USA) were transformed with a pPR-IBA2 plasmid (IBA Life Sciences, Germany) containing an expression cassette encoding a Pol6 DNA Polymerase—SpyCatcher fusion protein. The transformed cells were cultivated for protein expression according to the manufacturer's instructions and the fusion proteins were purified using a cobalt affinity column. The SpyCatcher-polymerase fusion was incubated with the 6:1 nanopores from Example 2 at a 1:1 molar ratio overnight at 4° C. in 3 mM SrCl₂. The polymerase-alpha hemolysin heptamer complex was then purified using size-exclusion chromatography.

A polymerase-pore-template complex was generated from the purified polymerase-alpha hemolysin heptamer complex as described in US 2017-0268052 and inserted onto a sequencing array as described in in PCT/US14/61853. Negatively charged tagged nucleotides were flowed onto the system in the presence of a buffer comprising 20 mM HEPES pH 8, 300 mM KGlu, 3 mM Mg²⁺ and a standard sequencing run was conducted. Aggregated data from the sequencing run was filtered for only pores that generated a high quality read (HQR) and the percentage of HQRs that showed evidence of template threading was calculated.

This experiment was repeated for a wide channel alpha hemolysin nanopore (Pore P-0304) and for two narrow channel alpha hemolysin nanopores that have D127G+D128K substitutions (Pores P-0411 and P-0414). As illustrated at FIG. 3, P-0304 had greater than 15% of pores exhibiting a threaded state, whereas P-0411 and P-0414 both had less than 2% of pores exhibiting a threaded state.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### SEQUENCE LISTING FREE TEXT

(Mature WT aHL; AAA26598)
SEQ ID NO: 1

| | |
|---|---|
| ADSDINIKTG TTDIGSNTTV KTGDLVTYDK ENGMHKKVFY SFIDDKNHNK | 50 |
| KLLVIRTKGT IAGQYRVYSE EGANKSGLAW PSAFKVQLQL PDNEVAQISD | 100 |
| YYPRNSIDTK EYMSTLTYGF NGNVTGDDTG KIGGLIGANV SIGHTLKYVQ | 150 |
| PDFKTILESP TDKKVGWKVI FNNMVNQNWG PYDRDSWNPV YGNQLFMKTR | 200 |
| NGSMKAADNF LDPNKASSLL SSGFSPDFAT VITMDRKASK QQTNIDVIYE | 250 |
| RVRDDYQLHW TSTNWKGTNT KDKWTDRSSE RYKIDWEKEE MTN | 293 |

(aHL Variant G2055; D13A + H35G + D127G + D128K + H144A + V149K)
SEQ ID NO: 2

| | |
|---|---|
| ADSDINIKTG TTAIGSNTTV KTGDLVTYDK ENGMGKKVFY SFIDDKNHNK | 50 |
| KLLVIRTKGT IAGQYRVYSE EGANKSGLAW PSAFKVQLQL PDNEVAQISD | 100 |
| YYPRNSIDTK EYMSTLTYGF NGNVTGGKTG KIGGLIGANV SIGATLKYKQ | 150 |
| PDFKTILESP TDKKVGWKVI FNNMVNQNWG PYDRDSWNPV YGNQLFMKTR | 200 |
| NGSMKAADNF LDPNKASSLL SSGFSPDFAT VITMDRKASK QQTNIDVIYE | 250 |
| RVRDDYQLHW TSTNWKGTNT KDKWTDRSSE RYKIDWEKEE MTN | 293 |

(aHL Variant G2097; H35G + N47K + D127G + D128K + H144A + V149K)
SEQ ID NO: 3

| | |
|---|---|
| ADSDINIKTG TTDIGSNTTV KTGDLVTYDK ENGMGKKVFY SFIDDKKHNK | 50 |
| KLLVIRTKGT IAGQYRVYSE EGANKSGLAW PSAFKVQLQL PDNEVAQISD | 100 |
| YYPRNSIDTK EYMSTLTYGF NGNVTGGKTG KIGGLIGANV SIGATLKYKQ | 150 |
| PDFKTILESP TDKKVGWKVI FNNMVNQNWG PYDRDSWNPV YGNQLFMKTR | 200 |
| NGSMKAADNF LDPNKASSLL SSGFSPDFAT VITMDRKASK QQTNIDVIYE | 250 |
| RVRDDYQLHW TSTNWKGTNT KDKWTDRSSE RYKIDWEKEE MTN | 293 |

(aHL Variant G1742; H35G + N47K + E111N + M113A + D127G + D128K +
T129G + K131G + H144A + K147N + V149K)
SEQ ID NO: 4

| | |
|---|---|
| ADSDINIKTG TTDIGSNTTV KTGDLVTYDK ENGMGKKVFY SFIDDKKHNK | 50 |
| KLLVIRTKGT IAGQYRVYSE EGANKSGLAW PSAFKVQLQL PDNEVAQISD | 100 |
| YYPRNSIDTK NYASTLTYGF NGNVTGGKGG GIGGLIGANV SIGATLNYKQ | 150 |
| PDFKTILESP TDKKVGWKVI FNNMVNQNWG PYDRDSWNPV YGNQLFMKTR | 200 |
| NGSMKAADNF LDPNKASSLL SSGFSPDFAT VITMDRKASK QQTNIDVIYE | 250 |
| RVRDDYQLHW TSTNWKGTNT KDKWTDRSSE RYKIDWEKEE MTN | 293 |

(aHL Variant G1678; H35G + E111N + M113A + D127G + D128G +
T129G + K131G+ K147N)
SEQ ID NO: 5

| | |
|---|---|
| ADSDINIKTG TTDIGSNTTV KTGDLVTYDK ENGMGKKVFY SFIDDKNHNK | 50 |
| KLLVIRTKGT IAGQYRVYSE EGANKSGLAW PSAFKVQLQL PDNEVAQISD | 100 |
| YYPRNSIDTK NYASTLTYGF NGNVTGGGGG GIGGLIGANV SIGATLNYVQ | 150 |
| PDFKTILESP TDKKVGWKVI FNNMVNQNWG PYDRDSWNPV YGNQLFMKTR | 200 |
| NGSMKAADNF LDPNKASSLL SSGFSPDFAT VITMDRKASK QQTNIDVIYE | 250 |
| RVRDDYQLHW TSTNWKGTNT KDKWTDRSSE RYKIDWEKEE MTN | 293 |

(aHL Variant G639; H35G + N47K + H144A + V149K)
SEQ ID NO: 6

| | |
|---|---|
| ADSDINIKTG TTDIGSNTTV KTGDLVTYDK ENGMGKKVFY SFIDDKKHNK | 50 |
| KLLVIRTKGT IAGQYRVYSE EGANKSGLAW PSAFKVQLQL PDNEVAQISD | 100 |
| YYPRNSIDTK EYMSTLTYGF NGNVTGDDTG KIGGLIGANV SIGATLKYKQ | 150 |
| PDFKTILESP TDKKVGWKVI FNNMVNQNWG PYDRDSWNPV YGNQLFMKTR | 200 |
| NGSMKAADNF LDPNKASSLL SSGFSPDFAT VITMDRKASK QQTNIDVIYE | 250 |
| RVRDDYQLHW TSTNWKGTNT KDKWTDRSSE RYKIDWEKEE MTN | 293 |

(aHL Variant G1032; K8D)
SEQ ID NO: 7

| | |
|---|---|
| ADSDINIDTG TTDIGSNTTV KTGDLVTYDK ENGMHKKVFY SFIDDKNHNK | 50 |
| KLLVIRTKGT IAGQYRVYSE EGANKSGLAW PSAFKVQLQL PDNEVAQISD | 100 |
| YYPRNSIDTK EYMSTLTYGF NGNVTGDDTG KIGGLIGANV SIGHTLKYVQ | 150 |
| PDFKTILESP TDKKVGWKVI FNNMVNQNWG PYDRDSWNPV YGNQLFMKTR | 200 |
| NGSMKAADNF LDPNKASSLL SSGFSPDFAT VITMDRKASK QQTNIDVIYE | 250 |
| RVRDDYQLHW TSTNWKGTNT KDKWTDRSSE RYKIDWEKEE MTN | 293 |

(aHL Variant G2043; D128K + V149K)
SEQ ID NO: 8

| | |
|---|---|
| ADSDINIKTG TTDIGSNTTV KTGDLVTYDK ENGMHKKVFY SFIDDKNHNK | 50 |
| KLLVIRTKGT IAGQYRVYSE EGANKSGLAW PSAFKVQLQL PDNEVAQISD | 100 |
| YYPRNSIDTK EYMSTLTYGF NGNVTGDKTG KIGGLIGANV SIGHTLKYKQ | 150 |
| PDFKTILESP TDKKVGWKVI FNNMVNQNWG PYDRDSWNPV YGNQLFMKTR | 200 |
| NGSMKAADNF LDPNKASSLL SSGFSPDFAT VITMDRKASK QQTNIDVIYE | 250 |
| RVRDDYQLHW TSTNWKGTNT KDKWTDRSSE RYKIDWEKEE MTN | 293 |

(WT aHL DNA)
SEQ ID NO: 9

| | |
|---|---|
| ATGGCAGATC TCGATCCCGC GAAATTAATA CGACTCACTA TAGGGAGGCC | 50 |
| ACAACGGTTT CCCTCTAGAA ATAATTTTGT TTAACTTTAA GAAGGAGATA | 100 |
| TACAAATGGA TTCAGATATT AATATTAAAA CAGGTACAAC AGATATTGGT | 150 |
| TCAAATACAA CAGTAAAAAC TGGTGATTTA GTAACTTATG ATAAAGAAAA | 200 |
| TGGTATGCAT AAAAAAGTAT TTTATTCTTT TATTGATGAT AAAAATCATA | 250 |
| ATAAAAAATT GTTAGTTATT CGTACAAAAG GTACTATTGC AGGTCAATAT | 300 |
| AGAGTATATA GTGAAGAAGG TGCTAATAAA AGTGGTTTAG CATGGCCATC | 350 |
| TGCTTTTAAA GTTCAATTAC AATTACCTGA TAATGAAGTA GCACAAATTT | 400 |
| CAGATTATTA TCCACGTAAT AGTATTGATA CAAAAGAATA TATGTCAACA | 450 |
| TTAACTTATG GTTTTAATGG TAATGTAACA GGTGATGATA CTGGTAAAAT | 500 |
| TGGTGGTTTA ATTGGTGCTA ATGTTTCAAT TGGTCATACA TTAAAATATG | 550 |
| TACAACCAGA TTTTAAAACA ATTTTAGAAA GTCCTACTGA TAAAAAAGTT | 600 |
| GGTTGGAAAG TAATTTTTAA TAATATGGTT AATCAAAATT GGGGTCCTTA | 650 |
| TGATCGTGAT AGTTGGAATC CTGTATATGG TAATCAATTA TTTATGAAAA | 700 |
| CAAGAAATGG TTCTATGAAA GCAGCTGATA ATTTCTTAGA TCCAAATAAA | 750 |
| GCATCAAGTT TATTATCTTC AGGTTTTTCT CCTGATTTTG CAACAGTTAT | 800 |
| TACTATGGAT AGAAAAGCAT CAAAACAACA AACAAATATT GATGTTATTT | 850 |
| ATGAACGTGT AAGAGATGAT TATCAATTAC ATTGGACATC AACTAATTGG | 900 |
| AAAGGTACAA ATACTAAAGA TAAATGGACA GATAGAAGTT CAGAAAGATA | 950 |
| TAAAATTGAT TGGGAAAAAG AAGAAATGAC AAATGGTCTC AGCGCTTGGA | 1000 |
| GCCACCCGCA GTTCGAAAAA TAA | 1023 |

### CITATION LIST

Akeson et al., Microsecond timescale discrimination among polycytidylic acid, polyadenylic acid, and polyuridylic acid as homopolymers or as segments within single RNA molecules, Biophys. J. (1999) 77:3227-3233.

Aksimentiev and Schulten, Imaging α-Hemolysin with Molecular Dynamics: Ionic Conductance, Osmotic Permeability, and the Electrostatic Potential Map, Biophysical Journal (2005) 88:3745-3761.

Bhattacharya et al., Rectification of the Current in α-Hemolysin Pore Depends on the Cation Type: The Alkali Series Probed by Molecular Dynamics Simulations and Experiments, The Journal of Physical Chemistry (2011), Vol. 115, Issue 10, pp. 4255-4264.

Butler et al., Single-molecule DNA detection with an engineered MspA protein nanopore, PNAS (2008) 105(52): 20647-20652.

Chen et al., Fusion Protein Linkers: Property, Design and Functionality, Advanced Drug Delivery Reviews, 15 Oct. 2013, Vol. 65, Issue 10, pp. 1357-1369.

Hammerstein et al., Subunit dimers of α-hemolysin expand the engineering toolbox for protein Nanopores, Journal of Biological Chemistry, Vol. 286, Issue 16, pp. 14324-34.

Howorka et al., Sequence-specific detection of individual DNA strands using engineered nanopores, Nat. Biotechnol., 19 (2001a), pp. 636-639.

Howorka et al., Kinetics of duplex formation for individual DNA strands within a single protein nanopore, Proc. Natl. Acad. Sci. USA, 98 (2001b), pp. 12996-13001.

Kasianowicz et al., Nanometer-scale pores: potential applications for analyte detection and DNA characterization, Proc. Natl. Acad. Sci. USA (1996) 93:13770-13773.

Korchev et al., Low Conductance States of a Single Ion Channel are not ‘Closed’, J. Membrane Biol. (1995) 147:233-239.

Krasilnikov and Sabirov, Ion Transport Through Channels Formed in Lipid Bilayers by Staphylococcus aureus Alpha-Toxin, Gen. Physiol. Biophys. (1989) 8:213-222.

Meller et al., Voltage-driven DNA translocations through a nanopore, Phys. Rev. Lett., 86 (2001), pp. 3435-3438.

Movileanu et al., Detecting protein analytes that modulate transmembrane movement of a polymer chain within a single protein pore, Nat. Biotechnol., 18 (2000), pp. 1091-1095.

Nakane et al., A Nanosensor for Transmembrane Capture and Identification of Single Nucleic Acid Molecules, Biophys. J. (2004) 87:615-621.

Noskov et al., Ion Permeation through the α-Hemolysin Channel: Theoretical Studies Based on Brownian Dynamics and Poisson-Nernst-Plank Electrodiffusion Theory, Biophysical Journal (2004), Vol. 87, Issue 4, pp. 2299-2309

Rhee and Burns, Nanopore sequencing technology: nanopore preparations, TRENDS in Biotech. (2007) 25 (4): 174-181.

Song et al., Structure of Staphylococcal α-Hemolysin, a Heptameric Transmembrane Pore, Science (1996) 274:1859-1866.

Stoddart et al., Single-nucleotide discrimination in immobilized DNA oligonucleotides with a biological nanopore, Proceedings of the National Academy of Sciences of the United States of America (2009), Vol. 106, Issue 19, pp. 7702-7707.

The entirety of each patent, patent application, publication, document, GENBANK sequence, website and other published material referenced herein hereby is incorporated by reference, including all tables, drawings, and figures. All patents and publications are herein incorporated by reference to the same extent as if each was specifically and individually indicated to be incorporated by reference. Citation of the above patents, patent applications, publications and documents is not an admission that any of the foregoing is pertinent prior art, nor does it constitute any admission as to the contents or date of these publications or documents. All patents and publications mentioned herein are indicative of the skill levels of those of ordinary skill in the art to which the invention pertains.

1. A polypeptide comprising a variant narrow channel alpha-hemolysin subunit,
wherein said variant narrow channel alpha hemolysin subunit has at least the following characteristics:
(a) at least 75% identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8;
(b) a D127G substitution relative to SEQ ID NO: 1;
(c) a D128K substitution relative to SEQ ID NO: 1; and
(d) one or more of the following:
(d1) an amino acid at a position corresponding to E111 of SEQ ID NO: 1 that has a sidechain that is longer than the side chain of asparagine,
(d2) an amino acid at a position corresponding to K147 of SEQ ID NO: 1 that has a sidechain that is longer than the side chain of asparagine, and/or
(d3) an amino acid at a position corresponding to M113 of SEQ ID NO: 1 that has a sidechain that is longer than the side chain of alanine.

2. The polypeptide of claim 1, wherein the variant narrow channel alpha hemolysin subunit has at least 80%, at least 85%, at least 90%, at least 95% or more identity to at least one of SEQ ID NO: 1, SEQ ID NO:2, SEQ ID NO: 3, SEQ ID NO:4, SEQ ID NO:5, SEQ ID NO:6, SEQ ID NO:7, and SEQ ID NO:8.

3. The polypeptide of claim 1, wherein the amino acid at the position corresponding to E111 is selected from the group consisting of glutamic acid, lysine, arginine, and glutamine.

4. The polypeptide of claim 1, wherein the amino acid at the position corresponding to E111 is selected from the group consisting of glutamic acid and lysine.

5. The polypeptide of claim 1, wherein the amino acid residue corresponding to E111 is glutamic acid.

6. The polypeptide of claim 1, wherein the amino acid at the position corresponding to K147 is selected from the group consisting of glutamic acid, lysine, arginine, and glutamine.

7. The polypeptide of claim 1, wherein the amino acid at the position corresponding to K147 is selected from the group consisting of glutamic acid and lysine.

8. The polypeptide of claim 1, wherein the amino acid at the position corresponding to K147 is lysine.

9. The polypeptide of claim 1, wherein the amino acid at the position corresponding to M113 is selected from the group consisting of leucine, isoleucine, valine, or methionine.

10. The polypeptide of claim 1, wherein the amino acid at the position corresponding to M113 is methionine.

11. A polypeptide comprising an amino acid sequence selected from the group consisting of:
(a) an amino acid sequence having at least 75% identity to SEQ ID NO: 1, wherein said amino acid sequence comprises
(a1) a D127G and a D128K substitution relative to SEQ ID NO: 1, and
(a2) each of E111, M113, and K147 of SEQ ID NO: 1;
(b) an amino acid sequence having at least 75% identity to SEQ ID NO: 2, wherein said amino acid sequence comprises each of G127, K128, E111, M113, and K147 of SEQ ID NO: 2;
(c) an amino acid sequence having at least 75% identity to SEQ ID NO: 3, wherein said amino acid sequence comprises each of G127, K128, E111, M113, and K147 of SEQ ID NO: 3;
(d) an amino acid sequence having at least 75% identity to SEQ ID NO: 4, wherein said amino acid sequence comprises
(d1) each of G127 and K128 of SEQ ID NO: 4,
(d2) an N111E substitution relative to SEQ ID NO: 4,
(d3) an N147K substitution relative to SEQ ID NO: 4, and
(d4) an A113M substitution relative to SEQ ID NO: 4;
(e) an amino acid sequence having at least 75% identity to SEQ ID NO: 5, wherein said amino acid sequence comprises:
(e1) G127 of SEQ ID NO: 5,
(e2) a G128K substitution relative to SEQ ID NO: 5,
(e3) an N111E substitution relative to SEQ ID NO: 5,
(e4) an N147K substitution relative to SEQ ID NO: 5, and
(e5) an A113M substitution relative to SEQ ID NO: 5;
(f) an amino acid sequence having at least 75%, identity to SEQ ID NO: 6, wherein the amino acid sequence comprises:
(f1) a D127G and a D128K substitution relative to SEQ ID NO: 6,
(f2) each of E111, K147, and M113 of SEQ ID NO: 6;
(g) an amino acid sequence having at least 75%, identity to SEQ ID NO: 7, wherein the amino acid sequence comprises:
(g1) a D127G and a D128K substitution relative to SEQ ID NO: 7, and
(g2) each of E111, M113, and K147 of SEQ ID NO: 7; and
(h) an amino acid sequence having at least 75%, identity to SEQ ID NO: 8, wherein the amino acid sequence comprises:
(h1) a D127G and a D128K substitution relative to SEQ ID NO: 8, and
(h2) each of E111, M113, and K147 of SEQ ID NO: 8.

12. The polypeptide of claim 11, wherein the amino acid sequence has at least 80%, at least 85%, at least 90%, at least 95% or more identity to at least one of SEQ ID NO: 1, SEQ ID NO:2, SEQ ID NO: 3, SEQ ID NO:4, SEQ ID NO:5, SEQ ID NO:6, SEQ ID NO:7, and SEQ ID NO:8.

13. A narrow channel alpha-hemolysin nanopore comprising at least 1 polypeptide according to claim 1.

14. The narrow channel alpha-hemolysin nanopore of claim 13, wherein the nanopore comprises at least 6 variant narrow channel alpha-hemolysin subunits comprising a D127G and a D128K substitution relative to SEQ ID NO: 1.

15. The narrow channel alpha-hemolysin nanopore of claim 14, wherein the narrow channel alpha hemolysin nanopore is a 6:1 nanopore and the “1” component is attached to a DNA polymerase.

16. A system for performing nucleic acid sequencing-by-synthesis (SBS), the system comprising:
(a) a chip comprising a plurality of sensing electrodes;
(b) an electrochemically resistive barrier disposed on a surface of the chip, wherein the barrier has a cis side and a trans side;
(c) a first electrolyte solution on the cis side of the barrier;
(d) a second electrolyte solution on the trans side of the barrier;
(e) a plurality of narrow channel alpha hemolysin nanopores according to claim 13, wherein the narrow channel alpha hemolysin nanopores are disposed in the barrier such that a channel of the narrow channel alpha hemolysin nanopores permits ion exchange between the first electrolyte solution and the second electrolyte solution, and wherein at least a portion of the narrow channel alpha hemolysin nanopores are close enough to one of the sensing electrodes that the sensing electrode can detect at least one characteristic of an electrical current flowing through the channel of the nanopore;
(f) a computer system in electronic communication with the sensing electrodes, wherein the computing system is adapted to record the characteristic of the electrical current flowing through the nanopore that is detected by the sensing electrode;
(g) a nucleic acid polymerase associated with the nanopore on the cis side of the barrier, wherein the nucleic acid polymerase is capable of catalyzing a template-dependent nucleic acid amplification reaction in the first electrolyte solution; and
(f) a set of nucleoside-5 [symbol] -oligophosphates disposed in the first electrolyte solution, the set including at least a polymer-tagged adenosine nucleoside-5 [symbol] -oligophosphate, a polymer-tagged guanine nucleoside-5 [symbol] -oligophosphate, a polymer-tagged cytosine nucleoside-5 [symbol] -oligophosphate, and either a polymer-tagged thymidine nucleoside-5 [symbol] -oligophosphate or a polymer-tagged uracil nucleoside-5 [symbol] -oligophosphate, wherein each of the polymer-tagged nucleoside-5 [symbol] -oligophosphates is the nucleoside-5 [symbol] -oligophosphate.

17. A sequencing-by-synthesis (SBS) method of sequencing a template nucleic acid, the method comprising:
providing a system according claim 16 having a plurality of active nanopore sequencing complexes, each active nanopore sequencing complex comprising:
at least one of the sensing electrodes;
one of the nanopores inserted in the barrier in proximity to the sensing electrode, wherein a current is flowing through the nanopore and a characteristic of the current is detected by the sensing electrode;
the nucleic acid polymerase associated with the nanopore; and
the template nucleic acid complexed with the nucleic acid polymerase;
at the active nanopore sequencing complexes, incorporating the tagged nucleoside-5 [symbol] -oligophosphates into a complementary nucleic acid of the template nucleic acid by a template-dependent nucleic acid amplification reaction catalyzed by the nucleic acid polymerase, wherein the polymer tag of the tagged nucleoside-5 [symbol] -oligophosphate moves into or in proximity to the channel of the nanopore as the tagged nucleoside-5 [symbol] -oligophosphate is incorporated into the complementary nucleic acid, and wherein movement of the polymer tag into or in proximity to the channel changes the characteristic of the current flowing through the nanopore;
detecting the change in the characteristic of the current flowing through the nanopore caused by the polymer tags with the sensing electrode and recording the change on the computer system; and
correlating each recorded change to one of the tagged nucleoside-5 [symbol] -oligophosphates, thereby generating a sequence of the complementary nucleic acid generated at that electrode.


SEQUENCE LISTING (9 sequences)

SEQ ID NO: 1 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 2 - protein, 293 aa, synthetic construct
  REGION 1..293: note = aHL Variant G2055
ADSDINIKTGTTAIGSNTTVKTGDLVTYDKENGMGKKVFYSFIDDKNHNKKLLVIRTKGTIAGQYRVYSEEGANKSGLAWPSAFKVQLQLPDNEVAQISDYYPRNSIDTKEYMSTLTYGFNGNVTGGKTGKIGGLIGANVSIGATLKYKQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 3 - protein, 293 aa, synthetic construct
  REGION 1..293: note = aHL Variant G2097
ADSDINIKTGTTDIGSNTTVKTGDLVTYDKENGMGKKVFYSFIDDKKHNKKLLVIRTKGTIAGQYRVYSEEGANKSGLAWPSAFKVQLQLPDNEVAQISDYYPRNSIDTKEYMSTLTYGFNGNVTGGKTGKIGGLIGANVSIGATLKYKQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 4 - protein, 293 aa, synthetic construct
  REGION 1..293: note = aHL Variant G1742
ADSDINIKTGTTDIGSNTTVKTGDLVTYDKENGMGKKVFYSFIDDKKHNKKLLVIRTKGTIAGQYRVYSEEGANKSGLAWPSAFKVQLQLPDNEVAQISDYYPRNSIDTKNYASTLTYGFNGNVTGGKGGGIGGLIGANVSIGATLNYKQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - protein, 293 aa, synthetic construct
  REGION 1..293: note = aHL Variant G1678
ADSDINIKTGTTDIGSNTTVKTGDLVTYDKENGMGKKVFYSFIDDKNHNKKLLVIRTKGTIAGQYRVYSEEGANKSGLAWPSAFKVQLQLPDNEVAQISDYYPRNSIDTKNYASTLTYGFNGNVTGGGGGGIGGLIGANVSIGATLNYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 6 - protein, 293 aa, synthetic construct
  REGION 1..293: note = aHL Variant G639
ADSDINIKTGTTDIGSNTTVKTGDLVTYDKENGMGKKVFYSFIDDKKHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGATLKYKQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 7 - protein, 293 aa, synthetic construct
  REGION 1..293: note = aHL Variant G1032
ADSDINIDTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 8 - protein, 293 aa, synthetic construct
  REGION 1..293: note = aHL Variant G2043
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDKTGKIGGLIGANVSIGHTLKYKQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 9 - DNA, 1023 bp, Staphylococcus aureus
  source 1..1023: mol_type = genomic DNA
atggcagatctcgatcccgcgaaattaatacgactcactatagggaggccacaacggtttccctctagaaataattttgtttaactttaagaaggagatatacaaatggattcagatattaatattaaaacaggtacaacagatattggttcaaatacaacagtaaaaactggtgatttagtaacttatgataaagaaaatggtatgcataaaaaagtattttattcttttattgatgataaaaatcataataaaaaattgttagttattcgtacaaaaggtactattgcaggtcaatatagagtatatagtgaagaaggtgctaataaaagtggtttagcatggccatctgcttttaaagttcaattacaattacctgataatgaagtagcacaaatttcagattattatccacgtaatagtattgatacaaaagaatatatgtcaacattaacttatggttttaatggtaatgtaacaggtgatgatactggtaaaattggtggtttaattggtgctaatgtttcaattggtcatacattaaaatatgtacaaccagattttaaaacaattttagaaagtcctactgataaaaaagttggttggaaagtaatttttaataatatggttaatcaaaattggggtccttatgatcgtgatagttggaatcctgtatatggtaatcaattatttatgaaaacaagaaatggttctatgaaagcagctgataatttcttagatccaaataaagcatcaagtttattatcttcaggtttttctcctgattttgcaacagttattactatggatagaaaagcatcaaaacaacaaacaaatattgatgttatttatgaacgtgtaagagatgattatcaattacattggacatcaactaattggaaaggtacaaatactaaagataaatggacagatagaagttcagaaagatataaaattgattgggaaaaagaagaaatgacaaatggtctcagcgcttggagccacccgcagttcgaaaaataa